(19) United States
(12) Patent Application Publication
(10) Pub. No.: US 2015/0245782 A1
(43) Pub. Date: Sep. 3, 2015

(54) SYSTEMS AND METHODS FOR CAPACITANCE SENSING IN MEDICAL DEVICES

(71) Applicant: Covidien LP, Mansfield, MA (US)

(72) Inventors: Eric Morland, Erie, CO (US); Daniel Lisogurski, Boulder, CO (US); Christopher Meehan, Golden, CO (US); Sarah Hayman, Boulder, CO (US); Darshan Iyer, Superior, CO (US); Friso Schlottau, Lyons, CO (US)

(21) Appl. No.: 14/577,689

(22) Filed: Dec. 19, 2014

Related U.S. Application Data
(60) Provisional application No. 61/946,140, filed on Feb. 28, 2014.

(51) Int. Cl. A61B 5/053; A61B 5/1455; A61B 5/00

(52) U.S. Cl. CPC A61B 5/053; A61B 5/0095; A61B 5/14551

(57) ABSTRACT

Various methods and systems for the use of capacitance sensors within medical devices configured for patient monitoring are provided. The capacitance sensors are configured to measure a change in capacitance resulting from a material (e.g., human tissue, water, gel, cloth, etc.) placed near (e.g., close proximity to) the medical device and/or resulting from a material making physical contact with the medical device. In certain embodiments, the capacitance sensor may be utilized to detect whether one or more portions of the medical sensor are securely applied to the patient's tissue (e.g., sensor “on”) and/or may be utilized to detect whether one or more portions of the medical sensor fail to maintain secure contact with the patient's tissue (e.g., sensor “off”). Further, in certain embodiments, the capacitance sensor may be utilized to distinguish between one or more types of materials (e.g., human tissue, water-based materials, etc.).

### BACKGROUND

The present disclosure relates generally to medical devices and, more particularly, to the use of capacitance sensing in medical devices configured for patient monitoring.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. In certain patient monitoring contexts, devices based on photoplethymography techniques may be utilized. For example, a patient may be monitored with a sensor that includes optical sensing components that are applied to a patient, such as pulse oximetry devices or sensors, which may be appropriate for a wide variety of patients.

In certain other patient monitoring contexts, it may be desirable to ascertain various localized physiological parameters, such as parameters related to individual blood vessels or other discrete components of the vascular system. Examples of such parameters may include oxygen saturation, regional saturation, hemoglobin concentration, perfusion, and so forth, for an individual blood vessel. In one example, measurement of such localized parameters may be achieved via photoacoustic (PA) spectroscopy, which utilizes light directed into a patient's tissue to generate acoustic waves that may be detected and resolved to determine localized physiological information of interest. In particular, the light energy directed into the tissue may be provided at particular wavelengths that correspond to the absorption profile of one or more blood or tissue constituents of interest. The light absorbed by the constituent of interest results in a proportionate increase in the kinetic energy of the constituent (i.e., the constituent is heated), which results in the generation of acoustic waves. The acoustic waves may be detected and used to determine the amount of light absorption, and thus the quantity of the constituent of interest, in the illuminated region.

In such medical contexts, equipment associated with these patient monitoring techniques and devices are often applied directly onto a patient's tissue in various configurations and/or orientations. For example, sensors configured for photoacoustic techniques or photoplethysmography techniques may be applied to the ear, neck, arm, leg, or any tissue region of interest on the patient. Further, sensors used for regional oxygen saturation monitoring may be applied to the temple and forehead of the patient. In addition, different sensor configurations and/or orientations (e.g., folded, non-folded) may be used when the sensors are applied to the patient's tissue for different modes of monitoring (e.g., transmission mode, reflectance mode, etc.).

In certain situations, sensors that are improperly applied may trigger false alarms, raise equipment safety issues, waste healthcare personnel time, or may result in inaccurate measurements of physiological parameters related to the patient's tissue. For example, one or more portions of a sensor not securely adhered to human tissue may lose contact with the tissue (e.g., fall off), leading to inaccurate reporting of physiological parameters. As a further example, one or more portions of a sensor may be improperly applied to a material other than human tissue (e.g., water, gel, cloth), leading to false reporting of physiological parameters by a sensor unable to distinguish between human tissues and other types of materials. Further still, a sensor may be applied to the patient's tissue with an improper configuration and/or orientation (e.g., reflectance mode instead of transmission mode), or may be applied to an improper tissue location (e.g., finger instead of cartilage).

Accordingly, it may be beneficial to assess the sensors utilized in patient monitoring contexts to ensure that all parts of the sensors are properly applied to human tissue instead of other types of materials, and to ensure that all parts of the sensors securely maintain contact with the patient's tissue. Further, it may be beneficial to detect the configuration and/or orientation of the sensors to determine the mode of sensor operation and/or the type of tissue being monitored.

### BRIEF DESCRIPTION OF THE DRAWINGS

Advantages of the disclosed techniques may become apparent upon reading the following detailed description and upon reference to the drawings in which:

FIG. 1 is a front view of an embodiment of a patient monitoring system in accordance with an embodiment;

FIG. 2 is a block diagram of the patient monitoring system of FIG. 1, illustrating a capacitance sensor disposed on a medical sensor, where the capacitance sensor is communicatively coupled to a capacitance-to-digital convertor within a patient monitor;

FIG. 3 is a perspective view of the medical sensor of FIG. 2, illustrating a double-plated capacitance sensor disposed on a sensor body;

FIG. 4 is a cut away side-view of the double-plated capacitance sensor of FIG. 3, illustrating an electric field generated between the transmitter plate and the receiver plate of the double-plated capacitance sensor;

FIG. 5 is an example of a graph depicting capacitance levels of the capacitance sensor over a period of time, illustrating a response to an approaching human tissue, in accordance with an embodiment;

FIG. 6 is a perspective view of the medical sensor of FIG. 2, illustrating a plurality of double-plated capacitance sensors disposed on the sensor body, where the plurality of double-plated capacitance sensors are configured to detect when a portion of the sensor loses contact with the human tissue;

FIG. 7 is a perspective view of the medical sensor of FIG. 2, illustrating a plurality of double-plated capacitance sensors and a water capacitance sensor disposed on the sensor body, where the water capacitance sensor is configured to distinguish between water-based fluids and human tissue;

FIG. 8 is a perspective view of the medical sensor of FIG. 2 illustrating the double-plated capacitance sensor, where the double-plated capacitance sensor is configured to distinguish between a reflectance mode of sensor operation and a transmission mode of sensor operation;

FIG. 9 is a perspective view of the medical sensor of FIG. 8, depicting the medical sensor in an un-folded orientation configured for reflectance mode operation;

FIG. 10 is a perspective view of the medical sensor of FIG. 8, depicting the medical sensor in a folded orientation configured for transmission mode operation;

FIG. 11 is a perspective view of the medical sensor configured for regional oximetry, illustrating a plurality of single-plated capacitance sensors disposed on the sensor body, including a single-plate water capacitance sensor;

FIG. 12 is a simplified block diagram of a patient monitoring system configured to be used with a sensor for PA spectroscopy, in accordance with described aspects of the present disclosure; and

FIG. 13 is a flow chart of a method for calculating an average physiological parameter signal based at least in part on the capacitance signal provided by the capacitance sensors of FIGS. 1-12.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Provided herein are systems and methods for the use of capacitance sensing in conjunction with medical devices configured for patient monitoring. As discussed in greater detail below, the capacitance sensors are configured to measure a change in capacitance resulting from a material (e.g., human tissue, water, gel, cloth, etc.) placed near (e.g., in close proximity to) the medical device and/or resulting from a material making physical contact with the medical device. In particular, the techniques described may be used within one or more different medical monitoring contexts and techniques, such as, for example, within photoplethysmography techniques (e.g., pulse oximetry and/or regional oximetry techniques), and/or photoacoustic techniques. Accordingly, the systems and methods described may be used within any suitable medical sensor configured to obtain any desired medical parameter information such as, for example, tissue hydration, total hemoglobin, regional saturation, oxygen saturation of hemoglobin in arterial blood, the volume of individual blood pulsations supplying the tissue, the rate of blood pulsations corresponding to each heartbeat of a patient, or any other suitable physiological parameter.

As discussed in greater detail below, a patient monitor is configured to obtain a signal (e.g., a plethysmographic signal, an optical signal, a photoacoustic signal, etc.) from the medical sensor and determine one or more physiological parameters from the signal. One or more capacitance sensors may be disposed on the medical sensor and may be configured to measure a change in capacitance to provide a capacitance signal to the patient monitor. The capacitance signal may include information related to a measured change in capacitance. In certain embodiments, the capacitance sensor may be a double-plated capacitance sensor configured to generate an electric field between each of the two plates (e.g., a first transmit plate and a second receive plate). In particular, when a non-conductive material (e.g., human tissue) interferes with the generated field, the material may act as a dielectric that weakens the electric field and increases the capacitance. The change in capacitance may be measured and transmitted by the double-plated capacitance sensor to the patient monitor. In other embodiments, the capacitance sensor may be a single-plated capacitance sensor, which, when paired with a material in close proximity or contact, becomes a parallel plate capacitor. In other words, a material (e.g., human tissue in the case of a proper sensor application) acts as the second plate of the single-plated capacitance sensor, and the change in capacitance resulting from placing the material in close proximity or contact is measured and transmitted by the capacitance sensor to the patient monitor. Further, the change in capacitance is measured by the capacitance sensor, which may be utilized by the patient monitor to determine various types of information related to sensor application.

In certain embodiments, one or more capacitance sensors disposed on a medical sensor may be configured to detect the type of material (e.g., human tissue, air, water, gel, cloth, etc.) the medical sensor is applied to based on one or more capacitance measurements collected by the capacitance sensors. For example, a capacitance measurement may be associated with a type of material, and may be used by the patient monitoring system to distinguish between different types of materials, such as, for example, between human tissue and water. Accordingly, in such embodiments, the capacitance sensor may be utilized to detect the presence of human tissue in close proximity (e.g., near, touching, etc.) to the medical sensor, and may be utilized to turn the medical sensor “off” when it is not receiving physiological parameter information from human tissue or to avoid posting a physiological parameter when the sensor is not attached to the patient. Likewise, detection of the presence of human tissue may be used to maintain the medical sensor “on” and operating. In certain embodiments, the patient monitor may be configured to calculate an average physiological parameter signal from one or more portions of the physiological parameter signal where the medical sensor was “on” and properly operating.

In addition, in certain embodiments, one or more capacitance sensors disposed on a medical sensor may be configured to detect the orientation and/or configuration of the medical sensor to determine the mode of sensor operation (e.g., transmission mode, reflectance mode, etc.) based on the change in capacitance measured. In certain other embodiments, one or more capacitance sensors disposed on a medical sensor may be configured to detect if the medical sensor does not securely maintain contact with a patient's tissue based on a measured change in capacitance. For example, the capacitance sensor may be utilized to detect whether one or more portions of the medical sensor are not securely applied to the patient's tissue and/or may be utilized to detect whether one or more portions of the medical sensor fail to maintain secure contact with the patient's tissue. In such embodiments, the one or more capacitance sensors may be disposed along the perimeter of the medical sensor, such that the capacitance sensor may be able to detect a change in capacitance when any portion of the medical sensor loses contact with the patient's tissue.

Accordingly, the patient monitor may use the capacitance signal to detect the type of material (e.g., human tissue, water, gel, cloth, etc.) the medical sensor is applied to, to ensure that all parts of the sensor are properly applied to human tissue instead of other types of materials, to ensure that all parts of the sensor securely maintain contact with the patient's tissue, to detect the configuration and/or orientation of the medical sensor on the patient's tissue, and/or to determine the mode of operation of the medical sensor. Further, in certain embodiments, the patient monitor may be configured to respond to the determined information related to sensor application. For example, the patient monitor may indicate sensor “on” and sensor “off” conditions based on the sensor's proximity and/or contact with human tissue, may provide alerts based on emergency sensor application situations, may be configured to turn certain portions of a sensor “off” (e.g., disable or reduce laser power to ensure eye safe levels), or may change physiological parameter signal processing techniques based on a detected mode of operation. In certain embodiments, the patient monitor may indicate a poorly applied sensor (e.g., loose, not secure, etc.) and/or sensor conditions related to a partially applied sensor. Further, in certain embodiments, the patient monitor may be configured to aggregate and/or average one or more portions of a physiological parameter signal when the sensor was “on” based at least in part on the capacitance sensor output (e.g., capacitance signal).

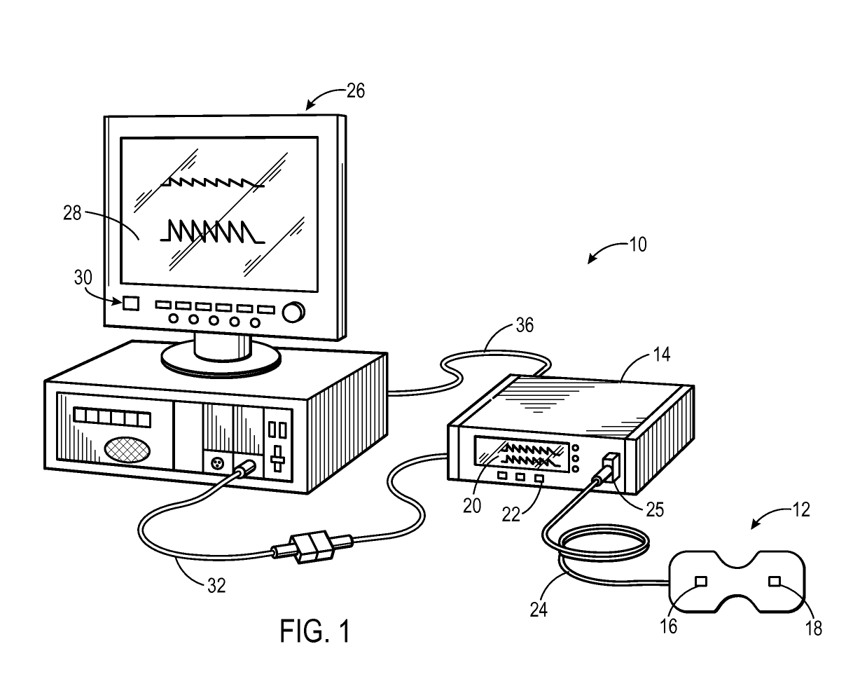

With the forgoing in mind, FIG. 1 depicts an embodiment of a patient monitoring system 10 that may be used in conjunction with a medical sensor 12. Although the depicted embodiments relate to photoplethysmography or pulse oximetry, the system 10 may be configured to obtain a variety of medical measurements with a suitable medical sensor. For example, the system 10 may additionally be configured to determine tissue hydration, total hemoglobin, regional saturation, or any other suitable physiological parameter. As noted, the system 10 includes the sensor 12 that is communicatively coupled to a patient monitor 14. The sensor 12 includes one or more emitters 16 and one or more detectors 18. The emitters 16 and detectors 18 of the sensor 12 are coupled to the monitor 14 via a cable 24 through a plug 25 coupled to a sensor port. Additionally, the monitor 14 includes a monitor display 20 configured to display information regarding the physiological parameters, information about the system, and/or alarm indications. The monitor 14 may include various input components 22, such as knobs, switches, keys and keypads, buttons, etc., to provide for operation and configuration of the monitor. The monitor 14 also includes a processor that may be used to execute code such as code for implementing the techniques discussed herein.

The monitor 14 may be any suitable monitor, such as a pulse oximetry monitor available from Covidien LP. Furthermore, to upgrade conventional operation provided by the monitor 14 to provide additional functions, the monitor 14 may be coupled to a multi-parameter patient monitor 26 via a cable 32 connected to a sensor input port or via a cable 36 connected to a digital communication port, or via an RF or optical wireless link. Alternatively, the techniques provided herein may be incorporated into one or more individual modules with plug-in connectivity to the multi-parameter patient monitor 26. Such modules may include connectors that allow the calculated physiological parameters to be sent to the host multi-parameter monitor. In addition, the monitor 14, or, alternatively, the multi-parameter patient monitor 26, may be configured to calculate physiological parameters and to provide a central display 28 for the visualization of information from the monitor 14 and from other medical monitoring devices or systems. The multi-parameter monitor 26 includes a processor that may be configured to execute code. The multi-parameter monitor 26 may also include various input components 30, such as knobs, switches, keys and keypads, buttons, etc., to provide for operation and configuration of the a multi-parameter monitor 26. In addition, the monitor 14 and/or the multi-parameter monitor 26 may be connected to a network to enable the sharing of information with servers or other workstations. In certain embodiments, the sensor 12 may be a wireless sensor 12. Accordingly, the wireless sensor 12 may establish a wireless communication with the patient monitor 14 and/or the multi-parameter patient monitor 26 using any suitable wireless standard. By way of example, the wireless module may be capable of communicating using one or more of the ZigBee standard, WirelessHART standard, Bluetooth standard, IEEE 802.11x standards, or MiWi standard. In embodiments in which the sensor 12 is configured for wireless communication, the strain relief features of the cable 24 may be housed in the sensor body 34.

As provided herein, the sensor 12 may be a sensor suitable for detection of one or more physiological parameters. The sensor 12 may include optical components (e.g., one or more emitters 16 and detectors 18). In one embodiment, the sensor 12 may be configured for photo-electric detection of blood and tissue constituents. For example, the sensor 12 may include pulse oximetry sensing functionality for determining the oxygen saturation of blood as well as other parameters from the plethysmographic waveform detected by the oximetry technique. An oximetry system may include a light sensor (e.g., sensor 12) that is placed at a site on a patient, typically a fingertip, toe, forehead or earlobe, or in the case of a neonate, across a foot. The sensor 12 may pass light using the emitter 16 through blood perfused tissue and photoelectrically sense the absorption of light in the tissue. For example, the monitor 14 may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. The light intensity or the amount of light absorbed may then be used to calculate the amount of the blood constituent (e.g., oxyhemoglobin) being measured and other physiological parameters such as the pulse rate and when each individual pulse occurs. Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption.

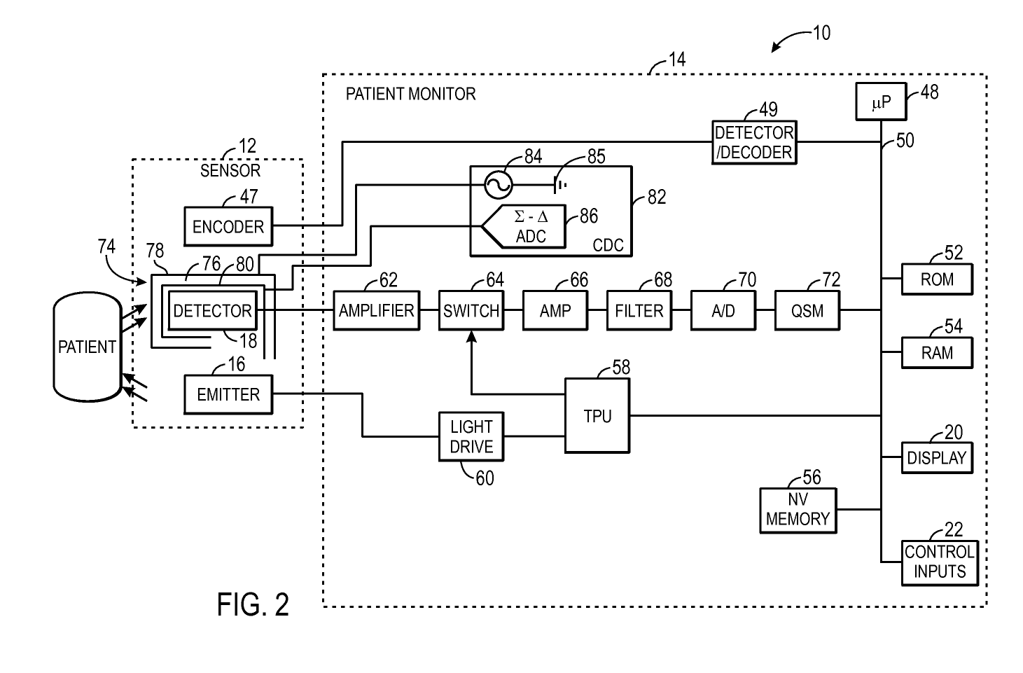

Turning to FIG. 2, a simplified block diagram of the medical system 10 is illustrated in accordance with an embodiment. As noted, the sensor 12 may include optical components in the forms of emitters 16 and detectors 18. The emitter 16 and the detector 18 may be arranged in a reflectance mode or transmission mode configuration with respect to one another. However, in embodiments in which the sensor 12 is configured for use on a patient's forehead (e.g. either alone or in conjunction with a hat or headband), the emitters 16 and detectors 18 may be in a reflectance configuration. Such sensors 12 may be used for pulse oximetry or regional saturation monitoring (e.g., INVOS® monitoring, as will be explained in detail with respect to FIG. 11). An emitter 16 may also be a light emitting diode, superluminescent light emitting diode, a laser diode or a vertical cavity surface emitting laser (VCSEL). An emitter 16 and detector 18 may also include optical fiber sensing elements. An emitter 16 may include a broadband or “white light” source, in which case the detector could include any of a variety of elements for selecting specific wavelengths, such as reflective or refractive elements, absorptive filters, dielectric stack filters, or interferometers. These kinds of emitters and/or detectors would typically be coupled to the sensor 12 via fiber optics. Alternatively, a sensor assembly 12 may sense light detected from the tissue is at a different wavelength from the light emitted into the tissue. Such sensors may be adapted to sense fluorescence, phosphorescence, Raman scattering, Rayleigh scattering and multi-photon events or photoacoustic effects in conjunction with the appropriate sensing elements.

In certain embodiments, the emitter 16 and detector 18 may be configured for pulse oximetry. It should be noted that the emitter 16 may be capable of emitting at least two wavelengths of light, e.g., red and infrared (IR) light, into the tissue of a patient, where the red wavelength may be between about 600 nanometers (nm) and about 700 nm, and the IR wavelength may be between about 800 nm and about 1000 nm. The emitter 16 may include a single emitting device, for example, with two LEDs, or the emitter 16 may include a plurality of emitting devices with, for example, multiple LEDs at various locations. In some embodiments, the LEDs of the emitter 16 may emit one or three or more different wavelengths of light. Such wavelengths may include a red wavelength of between approximately 620-700 nm (e.g., 660 nm), a far red wavelength of between approximately 690-770 nm (e.g., 730 nm), and an infrared wavelength of between approximately 860-940 nm (e.g., 900 nm). Other wavelengths may include, for example, wavelengths of between approximately 500-600 nm and/or 1000-1100 nm and/or 1200-1400 nm. Regardless of the number of emitting devices, light from the emitter 16 may be used to measure, for example, oxygen saturation, water fractions, hematocrit, or other physiologic parameters of the patient. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure. In another embodiment, two emitters 16 may be configured for use in a regional saturation technique. To that end, the emitters 16 may include two light emitting diodes (LEDs) that are capable of emitting at least two wavelengths of light, e.g., red or near infrared light. In one embodiment, the LEDs emit light in the range of 600 nanometers to approximately 1000 nm. In a particular embodiment, one LED is capable of emitting light at 730 nm and the other LED is capable of emitting light at 810 nm.

In any suitable configuration of the sensor 12, the detector 18 may be an array of detector elements that may be capable of detecting light at various intensities and wavelengths. In one embodiment, light enters the detector 18 after passing through the tissue of the patient. In another embodiment, light emitted from the emitter 16 may be reflected by elements in the patent's tissue to enter the detector 18. The detector 18 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by the detector 18, and when more light at a certain wavelength is reflected, more light of that wavelength is typically received from the tissue by the detector 18. The detector 18 may receive light that has not entered the tissue to be used as a reference signal. After converting the received light to an electrical signal, the detector 18 may send the signal to the monitor 14, where physiological characteristics may be calculated based at least in part on the absorption and/or reflection of light by the tissue of the patient.

In certain embodiments, the medical sensor 12 may also include an encoder 47 that may provide signals indicative of the wavelength of one or more light sources of the emitter 16, which may allow for selection of appropriate calibration coefficients for calculating a physical parameter such as blood oxygen saturation. The encoder 47 may, for instance, be a coded resistor, EEPROM or other coding devices (such as a capacitor, inductor, PROM, RFID, parallel resident currents, or a colorimetric indicator) that may provide a signal to a microprocessor 48 related to the characteristics of the medical sensor 12 to enable the microprocessor 48 to determine the appropriate calibration characteristics of the medical sensor 12. Further, the encoder 47 may include encryption coding that prevents a disposable part of the medical sensor 12 from being recognized by a microprocessor 48 unable to decode the encryption. For example, a detector/decoder 49 may translate information from the encoder 47 before it can be properly handled by the processor 48. In some embodiments, the encoder 47 and/or the detector/decoder 48 may not be present. In some embodiments, the encrypted information held by the encoder 47 may itself be transmitted via an encrypted data protocol to the detector/decoder 49, such that the communication between 47 and 49 is secured.

Signals from the detector 18 and/or the encoder 47 may be transmitted to the monitor 14. The monitor 14 may include one or more processors 48 coupled to an internal bus 50. Also connected to the bus may be a ROM memory 52, a RAM memory 54, non-volatile memory 56, a display 20, and control inputs 22. A time processing unit (TPU) 58 may provide timing control signals to light drive circuitry 60, which controls when the emitter 16 is activated, and if multiple light sources are used, the multiplexed timing for the different light sources. TPU 58 may also control the gating-in of signals from detector 18 through a switching circuit 64. These signals are sampled at the proper time, depending at least in part upon which of multiple light sources is activated, if multiple light sources are used. The received signal from the detector 18 may be passed through one or more amplifiers (e.g., amplifiers 62 and 66), a low pass filter 68, and an analog-to-digital converter 70 for amplifying, filtering, and digitizing the electrical signals from the sensor 12. The digital data may then be stored in a queued serial module (QSM) 72, for later downloading to RAM 54 as QSM 72 fills up. In an embodiment, there may be multiple parallel paths for separate amplifiers, filters, and A/D converters for multiple light wavelengths or spectra received.

Based at least in part upon the received signals corresponding to the light received by optical components of the pulse oximetry sensor 20, microprocessor 48 may calculate the oxygen saturation and/or heart rate using various algorithms, such as those employed by the Nellcor™ N-600x™ pulse oximetry monitor, which may be used in conjunction with various Nellcor™ pulse oximetry sensors, such as OxiMax™ sensors. In addition, the microprocessor 48 may calculate and/or display trend or parameter variability using various methods, such as those provided herein. These algorithms may employ certain coefficients, which may be empirically determined, and may correspond to the wavelengths of light used. The algorithms and coefficients may be stored in a ROM 52 or other suitable computer-readable storage medium and accessed and operated according to microprocessor 48 instructions. In one embodiment, the correction coefficients may be provided as a lookup table.

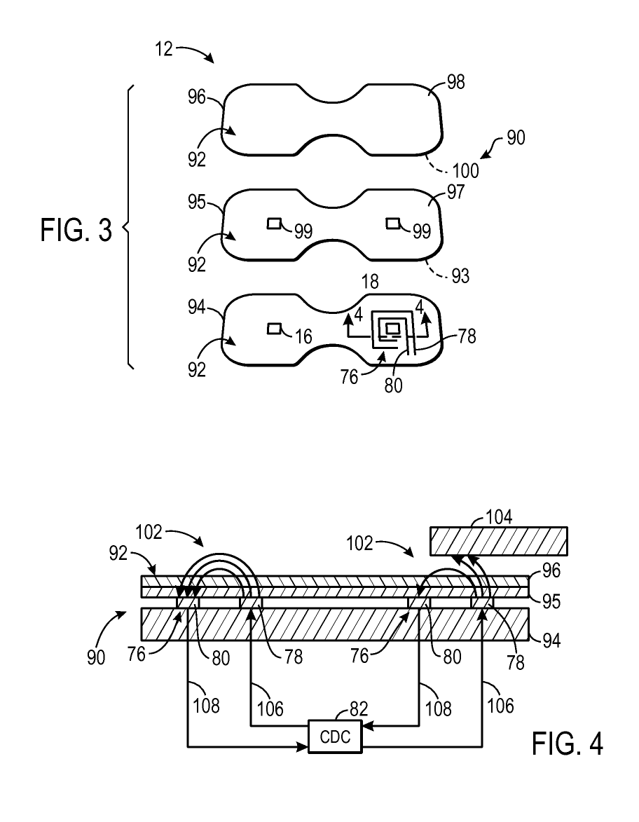

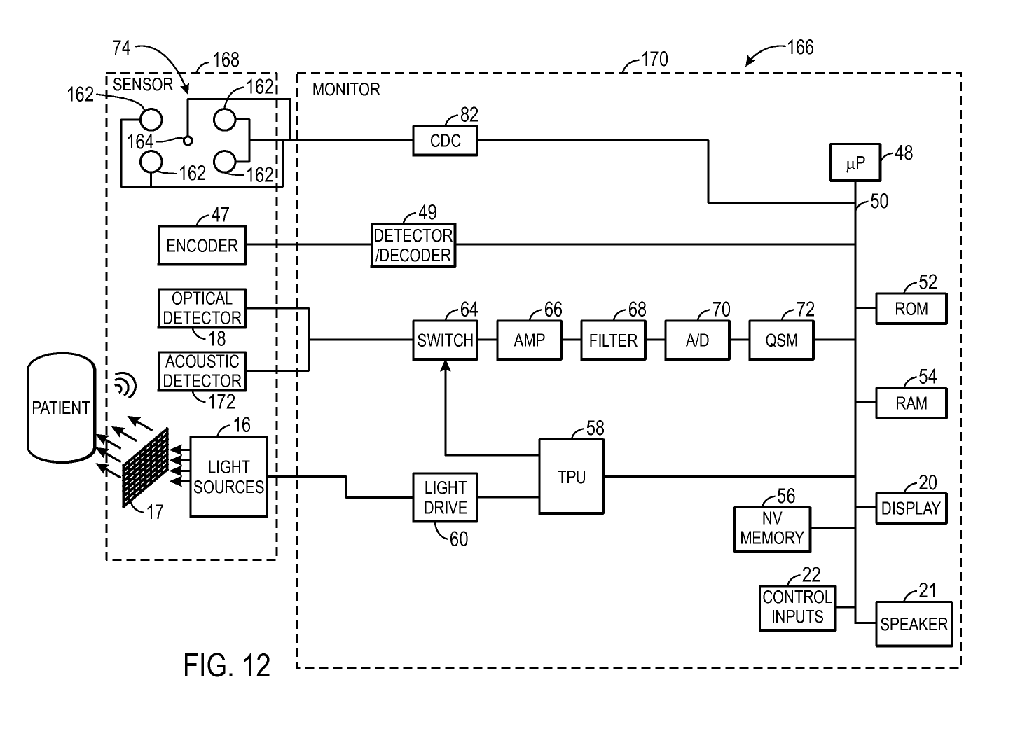

In certain embodiments, the medical sensor 12 may also include a capacitance sensor 76 configured to measure a change in capacitance resulting from a material, such as, for example, human tissue, water, gel, cloth, and so forth, being placed in close proximity and/or making physical contact with the sensor 12. Particularly, the illustrated embodiment of FIGS. 3-4 depicts a double-plated capacitance sensor 76, having a first plate 78 (e.g., transmission plate 78) and a second plate 80 (e.g., receiver plate 80) between which an electric field may be formed. In certain embodiments, the first plate 78 and the second plate 80 may be formed as a pattern of traces out of copper, carbon, silver, or other similar materials. The traces may be used to form a wide variety of capacitance sensors in a wide variety of shapes, such button capacitors, wheel capacitors, scroll-bar capacitors, switch capacitors, keypad capacitors, touchpad capacitors, and so forth. While the illustrated embodiment depicts a double-plated capacitance sensor 76, it should be noted that present techniques may be used in conjunction with a single-plated capacitance sensor, having a single or first plate 78 configured to function as a transmission plate 78 (as described in detail with respect to FIGS. 11-12). In such embodiments, an electric field may be formed between the first plate 78 (e.g., transmission plate 78) and a material brought within close proximity of the sensor 12, such that the material acts as the second plate 80 (e.g., receiver plate 80). For example, in a single-plate capacitance sensor having the transmission plate 78, human tissue placed within close proximity and/or having physical contact with the sensor 12 may be configured to function as the receiver plate 80.

In one embodiment, the capacitance sensor 76 on the sensor 12 may be coupled to provide a signal to separate signal processing circuitry, i.e., elements distinct from the circuitry used for processing the detector 18 signal. In one example, a capacitance-to-digital converter 82 (e.g., CDC 82) (shown in FIG. 2) is associated with the patient monitor 14. In certain embodiments, a plurality of capacitance sensors 76 disposed on the sensor 12 may be coupled to the CDC 82 (as depicted in FIGS. 2, 4, and 12). Further, the plurality of capacitance sensors 76 coupled to the CDC 82 on the patient monitor 14 may include different types of capacitance sensors 76 (e.g., button, wheel, switch, etc.) in different configurations (e.g., single-plated, double-plated, etc.). In certain embodiments, each plate of the single-plate or double-plate sensor design may be made up of one or more elements. For example, in some embodiments, two capacitance sensors 76 may be configured as one double-plate sensor or two single-plate sensor, providing for three modes of use configurable for software control. Indeed, the CDC 82 may include 2, 3, 4, 5, 6, 7, 8, 9, 10, 11, 12, 13, 14 or more capacitance input pins configured to receive a capacitance signal from the one or more capacitance sensors 76. In some embodiments, the CDC 82 may include an input multiplexor to select which capacitance sensor 76 is being monitored and/or several inputs may be monitored selectively by driving only one output (e.g., in the case of a two-plate sensor).

In some embodiments, the CDC 82 may be a one bit output based on comparing a capacitance to a threshold (e.g., configurable threshold, dynamic moving threshold, etc.) and/or may be a circuit where the output exceeds a threshold when certain conditions are met. The CDC 82 may determine relative changes in capacitance via a GPIO pin on a micro controller. For example, the GPIO pin may switch from high to low or low to high at a specific voltage. If a capacitor is charged by a fixed current source, the capacitance can be estimated by the time it takes for the GPIO pin to switch. Accordingly, switching the GPIO pin several times (charge/discharge timing) may allow for a more accurate estimate. In some embodiments, the microcontrollers may include internal peripherals that utilize a similar principle.

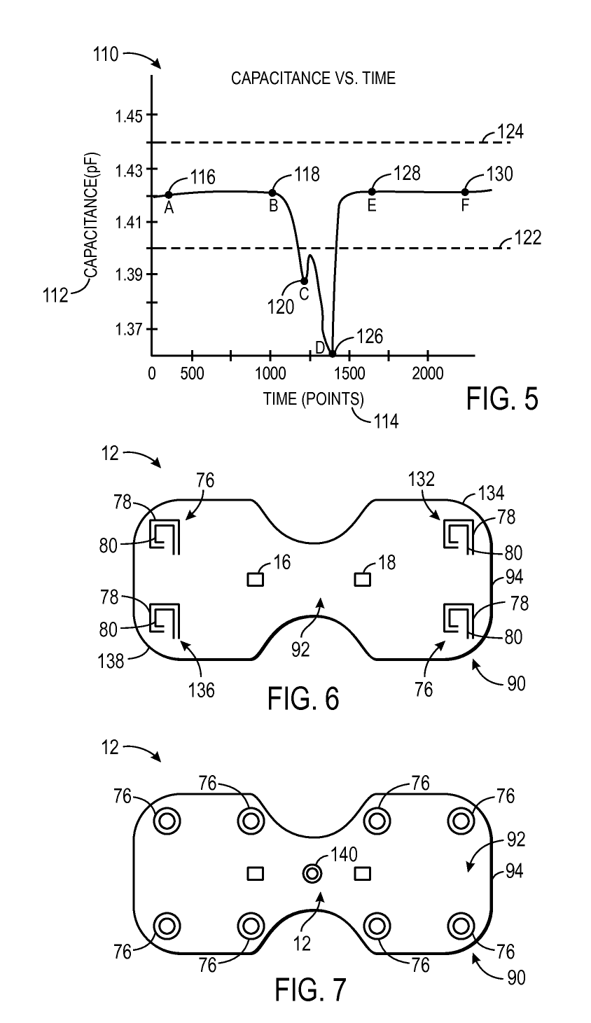

In particular, the CDC 82 includes an excitation source 84 and a sigma-delta Analog-to-Digital converter chip 86 (e.g., ADC chip 86). The excitation source 84 may be coupled to the transmission plate 78 on the double-plated capacitance sensor 76, and may be configured to provide an excitation signal to the transmission plate 78 and may additionally function as a ground 85. For example, the excitation source 84 may be an AC source configured to provide an AC excitation signal to the transmission plate 78. For example, the AC excitation signal may be a 250-kHz square wave to the transmission plate 78. An electric field may be formed between the transmission plate 78 and the receiver plate 80, such that the electric field extends from the transmission plate 78 and terminates at the receiver plate 80. The field strength at the receiver plate 80 is measured by the ADC chip 86 on the CDC 82. Specifically, the ADC chip receives the capacitance signal related to capacitance levels as an AC signal, and digitizes the information. In certain embodiments the digital values of the converted capacitance signal are provided to the microprocessor 48 for further processing, as described in detail below. In short, when a material (e.g., human tissue) interferes with the electrical field extending between the transmission plate 78 and the receiver plate 80 in a double-plated capacitance sensor 76, the CDC 82 measures a change in capacitance (e.g., a decrease in capacitance), as described further with respect to FIGS. 4-5.

The CDC 82 may be configured to continuously measure and receive a capacitance signal related to capacitance levels from each of the one or more capacitance sensors 76 or may intermittently receive a capacitance signal related to a chance in capacitance levels from any of the one or more capacitance sensors 76. In certain embodiments, the CDC 82 may be configured to continuously receive a capacitance signal from the capacitance sensor 76 on the sensor 12. With two or more capacitance sensors 76, the CDC 82 may be configured to continuously take sequential measurements of the capacitance from each capacitance sensor 76. For example, with two capacitance sensors 76, the CDC 82 may measure the capacitance at each capacitance sensor 76 with approximately 30 ms to 40 ms in between each measurement. Accordingly, the status of each capacitance sensor 76 may be detected essentially simultaneously even during sequential measurements. In certain embodiments, the CDC 82 may include a sequencer (not shown) configured to determine the order in which the plurality of capacitance sensors 76 are measured. In other embodiments, the CDC 82 may receive a capacitance signal in response to a measured change in capacitance levels at any one of the one or more capacitance sensors 76 on the sensor 12. In such embodiments, the sensor 12 may include an ACD chip 86 configured to measure the capacitance levels of the one or more capacitance sensors 76 to detect a change in capacitance. Further, the sensor 12 may additionally include a micro-controller configured to send one or more capacitance signals to the CDC 82 in response to a measured change in capacitance.

In another embodiment, the functions of the CDC 82 may be fulfilled by the one or more components used to process the incoming detector signal. For example, an analog signal from the capacitance sensor 76 may be digitized by the ADC 70 and provided to other circuitry, including amplifiers or filters, for further processing (e.g., to ROM 52 or RAM 54). Further, in certain embodiments, all or part of the signal processing pathway for the detector signal and the capacitance sensor may be disposed on or associated with the sensor 12 rather than being associated with the monitor 14.

The CDC 82 converts the capacitance input signal into digital values and may provide the digital values to the microprocessor 48 for further processing and/or analyzing using various algorithms. These algorithms may employ the use of various coefficients, constants, thresholds, limits, and/or values, which may be empirically determined or which may be pre-loaded and stored in the ROM 52 or other suitable computer-readable storage medium, such that it is accessed and operated according to the microprocessor 48, as will be described in detail below. In other embodiments, these coefficients, constants, thresholds, limits, and/or values may be provided as a lookup table accessible by the microprocessor 48. In yet other embodiments, these coefficients, constants, threshold, limits, and/or values are stored on the sensor 12 and may be used for processing of the capacitance signal before information is transmitted to the patient monitor 14.

In certain embodiments, such as during continuous measurements of the one or more capacitance sensors 76, the CDC 82 may be configured to continuously receive a capacitance signal related to the current status of each of the one or more capacitance sensors 76. The CDC 82 may convert the capacitance input signal to digital values, which may be used for further processing by the microprocessor 48 and/or may be stored in a memory of the patient monitor 14 for future processing. For example, when the sensor 12 is not active (e.g., not coupled to a material) or “off,” the capacitance value determined by the CDC 82 may be stored as an ambient or inactive value within the CDC 82 and/or within the QSM 72 for later downloading to the RAM 54 as the QSM 72 fills up. Further, when a material (e.g., human tissue, water, gel, cloth, plastic, etc.) comes within close proximity to and/or touches the capacitance sensor 76, the capacitance value (e.g., value representing the change in capacitance measured) may be stored as an active value within the CDC 82 or within the QSM 72. In addition, when the sensor 12 is poorly applied (e.g., the sensor 12 is loose, not securely attached to the tissue, etc.), the capacitance value determined by the CDC 82 may be also be stored as an active value within the CDC 82 or within the QSM 72. Specifically, the capacitance value generated when human tissue contacts the capacitance sensor may be stored as an active value, and may be used as a threshold for determining a sensor 12 condition, such as if the sensor 12 is “on” and/or if the sensor 12 is improperly applied (e.g., partial attachment, loose, etc.).

In certain embodiments, the converted capacitance signal (e.g., capacitance value) may be compared to an upper activation threshold and/or a lower activation threshold stored within the CDC 82 and/or the ROM 52. For example, the upper activation threshold and/or the lower activation threshold may be an upper dynamic threshold and/or a lower dynamic threshold that may change depending on historical information and/or on various dynamic parameters (e.g., drift, pulsatile amplitude, noise amplitude, variance, or any other parameter that may dynamically vary depending on the location of the sensor 12). The capacitive signal from a capacitance sensor can be expected to exhibit greater variance when contacting human tissue than when contacting an inert material or air, due to the physiologic activity of the human tissue. Thus, a measure of the variance or a dynamic property of the capacitive signal, such as the standard deviation of the signal, can be used as an indication that the sensor 12 is contacting human tissue, that the sensor 12 is not contacting human tissue, and/or that the sensor 12 is partially contacting human tissue. When the variance drops below a threshold, the system may determine that the sensor has lost contact with the patient. This variance threshold may be dynamic, such as a moving average, based on the recent history of sensor contact with the patient, to establish appropriate baselines for the individual patient. The capacitive signal may be analyzed in the frequency domain to identify the frequency content of the signal. For example, a sudden or significant change or drop in the frequency content may indicate that the sensor 12 is no longer exposed to physiological tissue. Other dynamic properties of the signal may be tracked and compared to a threshold for this purpose as well.

For example, the patient monitor 14 may compare the converted capacitance signal (e.g., capacitance value) to the upper dynamic threshold and/or the lower dynamic threshold by extracting any number of features from the capacitance signal and/or other signals provided to the patient monitor (e.g., optical signals) and then comparing the extracted features to pre-determined sensor 12 conditions. For example, in certain embodiments, the signals received by the patient monitor 14 may be initially filtered, conditioned, or pre-processed via one or more processing techniques, such as wavelet transforms or fast fourier transforms (FFT). In certain embodiments, the patient monitor 14 may initially process the received signals by blanking or removing noisy signals. In particular, one or more features may be extracted from the processed signals, such as, for example, a signal mean, a signal variance, a peak frequency, a ratio of power at heart rate (as well as harmonics) to all other power, a pulsatile amplitude, a ratio of pulsatile amplitude/mean signal level or more complex values derived from any algorithm. The extracted features may be provided as an input to a classifier (e.g., a neural net classifier, a statistically based classifier (maximum a priori (MAP) classifier), 1-NN (nearest neighbor classifier), fuzzy logic based classifier, or any other type of classifier) configured to determine the status of the sensor 12 (e.g., on, off, partially applied to the tissue, loose and/or insecure, faulty, etc.) based at least in part on the one or more of the extracted features. In certain embodiments, the classifier may have a confidence level or range that may be utilized to determine the accuracy of the determined sensor 12 status. In some situations, the classifier output may be processed through additional logic structures. For example, the classifier may determine that the sensor 12 has been “on” for approximately 10 minutes and “off” for approximately 3 seconds. In such situations, the patient monitor 14 may be configured to wait an additional period of time (e.g., approximately 10-20 seconds) before concluding that the sensor 12 is “off,” rather than reaching a conclusion based only on the information received for the 3 seconds. In this manner, the converted capacitance signal and one or more upper/lower dynamic thresholds may be utilized to determine the status of the sensor 12 (e.g., on, off, improperly applied to the tissue, etc.).

Accordingly, as noted above, in some embodiments, the current status (e.g., on, off, loose, etc.) of the sensor 12 may be determined by comparing the capacitance signal received to any simple threshold, dynamic threshold, and/or complex algorithm. Further, the current status of the sensor 12 may be determined by using current and/or historical capacitance signals measurements received. In addition, the capacitive signal may be analyzed in the time and/or frequency domain to identify the frequency content of the signal to determine the current status of the sensor 12. Further, the current status of the sensor 12 may be determined by utilizing one or more signals received from the sensor 12 in combination with the capacitance signal, as further described in detail with respect to FIGS. 9, 12, and 13.

Further, in certain embodiments, the active value (e.g., a measured change in capacitance below a lower activation threshold or above a higher activation threshold) may be compared to one or more dielectric constants, where each dielectric constant is associated with a different type of material and/or a different type of human tissue. The dielectric constants may vary for different parts of the body and for different types of materials (e.g., water, gel, plastic, cloth, etc.), and may be stored within the CDC 82, within a memory (e.g., a ROM memory 52, a RAM memory 54, non-volatile memory 56, etc.) on the patient monitor 14, and/or within a look-up table. Accordingly, an active value measured by one or more capacitance sensors 76 may correspond to a particular type of material (e.g., human tissue, water, gel, plastic cloth, etc.) and may be used to distinguish between one or more different types of materials and/or one or more different parts of the body. For example, the dielectric constant of human tissue varies approximately above 30 K (e.g., skin between approximately 33K-44K, eye between approximately 60K-67K, brain between approximately 40K-60K, etc.), while the dielectric constant of other materials (e.g., glass, plastics, metal, organic materials, paper, etc.) varies approximately between 1K-10K. Accordingly, a converted capacitance signal (e.g., digital value) below a lower activation threshold corresponds to an active value, and the patient monitor 14 may compare the active value to one or more stored dielectric constants to determine the type of material and/or the type of human tissue in contact or within close proximity to the medical sensor 12. In addition, certain embodiments of the capacitance sensor 76 are configured to distinguish between water and human tissue, which is described in greater detail with respect to FIGS. 7, 11, and 12.

In some embodiments, the CDC 82 may use a touch screen interface, such as the Touch Sense Input (TSI) peripheral included with one or more microcontrollers. The microcontrollers utilized may measure capacitance by the time to charge and discharge the plates of the sensor 12, and may compare the value measured to an internal reference capacitor. Accordingly, the electrical interaction between the body and the plate by the field may influence this frequency. The same processor may control the photoplethysmography or regional saturation hardware and execute algorithms to derive physiological parameters based on a combination of the optical signals and capacitance measurements. In some embodiments, the capacitance measurements may be done with different configurations to develop a profile of the capacitance at more than one excitation frequency. Selecting different frequencies may also mitigate interference between multiple devices on the subject. The system 10 may first analyze noise in the time or frequency domain to determine if the subject is in contact with another device or touch screen controller (e.g. on a phone or patient monitor 14) and select one or more appropriate frequencies, chirps, or use spread spectrum or frequency hopping techniques.

FIG. 3 is a perspective view of the medical sensor 12 of FIGS. 1-2, illustrating the double-plated capacitance sensor 76 disposed on the sensor 12. The sensor 12 includes a flexible sensor body 90 formed from a plurality of flexible layers disposed on a patient-facing surface 92. For example, the flexible layers may include a main nonconductive support layer 94, a non-conductive cover layer 95, and an adhesive layer 96.

The main nonconductive support layer 94 may support the emitter 16, the detector 18, the plurality of wires and/or leads associated with the sensor 12 and that couple to the emitter 16 and the detector 18, and the capacitance sensor 76 and any associated wires or leads. The main nonconductive support layer 94 may be constructed from any flexible polymeric or similar material that is approved or qualified for medical use and is capable of supporting various sensor components. Generally, the main nonconductive support layer 94 will be constructed from a polymeric material that is substantially non-transparent (i.e., opaque) with respect to wavelengths of ambient light that may interfere with the measurements performed by the bandage sensor 14. As an example, the main nonconductive support layer 94 may be constructed from an opaque (e.g., white) polypropylene that blocks wavelengths of light that may be used for pulse oximetry, such as infrared, near-infrared, visible, ultraviolet, or any combination thereof (e.g., between approximately 600 and 1400 nm). The main nonconductive support layer 94 may be disposed over the double-plated capacitor sensor 76, and other sensor components, and may be configured to act as an insulating surface. Particularly, the main nonconductive support layer 94 separates the electrical environment surrounding the capacitance sensor 76 from human tissue and/or other types of materials.

The sensor 12 may also include a non-conductive cover layer 95 that is coupled to the support layer 94 to sandwich the optical components and the double-plated capacitance sensor 76 between the layers 94 and 95. The bottom surface 93 of the cover layer 95 is coupled to the patient-facing surface 92 of the support layer 94. In certain embodiments, the cover layer 95 includes two or more optical windows 99 configured to allow the emitter 16 to emit wavelengths of light toward the patient's tissue and to allow the detector 18 to receive the light transmitted through the patient's tissue. The cover layer 95 may be constructed from materials with similar characteristics to those of the support layer 94. Alternatively, the support layer 94 may be folded over the optical components and the double-plated capacitance sensor 76 to form the cover layer 95.

In certain embodiments, the sensor 12 also includes an adhesive layer 96 disposed on the top surface 97 of the cover layer 95. Further, the adhesive layer 96 may be a thin transparent layer (e.g., transparent polyolefin, polyester or similar polymer) having an adhesive surface on a first surface 98 and a second surface 100 that couples to the cover layer 95. Accordingly, the non-conductive adhesive layer 96 may also be disposed over the capacitance sensor 76, and other sensor 12 components, providing additional insulation between the electrical environment around the capacitance sensor 76 and external materials, such as human tissues. Further, because the patient-contacting adhesive layer 96 covers the two or more optical windows 99 disposed on the cover layer 95, the non-conductive adhesive layer 96 may be transparent with respect to the wavelengths that are used for the particular implementation of the sensor 12.

As noted above, the double-plated capacitance sensor 76 may be disposed on the patient-facing surface 92 of the non-conductive support layer 94 such that the optical components of the sensors, the coupling wires, and the capacitance sensor are between the non-conductive support layer 94 and the non-conductive cover layer 95, which in turn is covered by the adhesive layer 96. That is, the electrical elements of the double-plated capacitance sensor 76 are not in direct contact with the patient when the sensor 12 is applied, but instead are shielded via one or more non-conductive layers. In certain embodiments, the double-plated capacitance sensor 76 includes the transmission plate 78 and the receiver plate 80, between which an electric field may be formed. In the illustrated embodiment, the transmission plate 78 and the receiver plate 80 are disposed on the sensor as a pattern of strips (e.g., traces) formed out of a conductive material, such as copper, carbon, silver, or other similar materials. It should be noted that while the present embodiment depicts the double-plated capacitance sensor 76 disposed around the detector 18, in other embodiments, the capacitance sensor 76 may be disposed anywhere on the patient-facing surface 92 of the support layer 94. Specifically, a plurality of capacitance sensors 76 may be disposed on the sensor body 90, as described in further detail with respect to FIGS. 6, 7, and 11.

FIG. 4 is a cut away side view of the double-plated capacitance sensor 76 of FIG. 3, illustrating an electric field 102 generated between the first plate 78 (e.g., transmission plate 78) and the second plate 80 (e.g., receiver plate 80). Further, the illustrated embodiment depicts the electric field 102 when interrupted by human tissue 104 (e.g., finger 104).

As noted above, the double-plated capacitance sensor 76 may be coupled to the CDC 82 on the patient monitor 12. The CDC 82 may provide an excitation signal 106 to the transmission plate 78 of the double-plated capacitance sensor 76 via the excitation source 84. In certain embodiments, the excitation signal 106 may be an alternating current (AC) signal at various frequencies. For example, the excitation signal 106 may be at a frequency between approximately 10 kHz to 200 kHz (e.g., 10 kHz, 50 kHz, 100 kHz, 200 kHz, 250 kHz, 300 kHz, and so forth). In response to the excitation signal 106, the electric field 102, such as an electric fringe field, may extend from the transmission plate 78 and terminate at the receiver plate 80. In certain situations, a material brought within close proximity and/or making physical contact with the sensor 12 may interfere with the electrical field 102 extending between the transmission plate 78 and the receiver plate 80. For example, in the illustrated embodiment, the human tissue 104 interferes with a portion of the electric field 102, interfering with the field strength of the electrical field 102 and leading to a decrease in capacitance levels. Accordingly, the double-plated capacitance sensor 76 may provide a capacitance signal 108 to the CDC 82 indicative of the change in capacitance (e.g., a decrease in capacitance levels). It should be noted that the type of material (e.g., type of human tissue, water, gel, plastic, cloth, metal, etc.) may have a direct correlation with the amount of change detected within the capacitance levels.

The CDC 82, disposed within the patient monitor 12, may receive the capacitance signal 108 from the receiver plate 80 of the double-plated capacitance sensor 76. The CDC 82 may be configured to convert the capacitance signal 108 from the AC signal to digital values, where the digital values are indicative of the measured changes in capacitance levels. The digital values may be stored within the memory of the patient monitor 12 and/or within the CDC 82 for future processing by the microprocessor 48. In certain embodiments, the CDC 82 may continuously provide the excitation signal 106 to each capacitance sensor 76 and continuously receive the capacitance signal 108 indicative of the capacitance levels of the capacitance sensor 76. As noted above, in embodiments with a plurality of capacitance sensors 76 (e.g., including double-plated capacitance sensors and/or single-plated capacitance sensors), the CDC 82 may sequentially provide the excitation signal 106 and receive the capacitance signal 108 from each capacitance sensor 76. In other embodiments, the CDC 82 may be configured to continuously provide the excitation signal 106 to each capacitance sensor 76, but may receive a capacitance signal 108 only in response to a change in capacitance levels.

Accordingly, the patient monitor 14 may be configured to detect changes in capacitance levels resulting from a material, such as human tissue 104, coming within close proximity and/or contact with the sensor 12. Further, the patient monitor 14 may be configured to detect changes in capacitance levels related to a particular capacitance sensor 76 disposed on the medical sensor 12, such that the patient monitor 14 may detect which one of the plurality of capacitance sensors 76 is within close proximity and/or contact with the material, such as human tissue 104. As such, the patient monitor 14 may detect periods of time during which the medical sensor 12 is active, “on,” and physically contacting the human tissue 104, and may detect periods of time during which the medical sensor 12 is non-active, “off,” and/or not contacting or within close proximity of the human tissue 104, as described in detail with respect to FIGS. 4-5. In some situations, the patient monitor 14 may additionally detect period of time during which the sensor 12 is poorly applied (e.g., loose or not securely attached) to the human tissue 104 and/or is a faulty sensor 12 that must be replaced.

FIG. 5 is an example of a graph 110 depicting capacitance levels 112 of a capacitance sensor 76 over a period of time 114, in response to the human tissue 104 approaching the medical sensor 12. As noted above, based at least in part on the capacitance signal 108 received from the capacitance sensor 76, the patient monitor 14 may be configured to detect periods of time during which the medical sensor 12 is “on,” such as periods of time during which the medical sensor 12 is within close proximity and/or contacting the human tissue 104. Likewise, based at least in part on the capacitance signal 108 received from the capacitance sensor 76, the patient monitor 14 may be configured to detect periods of time during which the medical sensor 12 is “off,” such as period of time during which the medical sensor 12 is not within close proximity and/or contacting the human tissue 104. Further, the patient monitor 14 may be configured to detect period of time during which the sensor 12 is improperly applied to the tissue 104, such as during the period of time when only portion of the sensor 12 is on the tissue 104.

In the illustrated embodiment, the graph 110 depicts measured capacitance signals 108 continuously received from the capacitance sensor 76 disposed on the medical sensor 12. Between a time point A 116 and a time point B 118, the capacitance signals 108 received by the CDC 82 may be indicative of ambient values, such as those measured during the time period the medical sensor 12 is “off,” and/or not within close proximity/contacting the human tissue 104. Further, at a time point C 120, the capacitance signals 108 received by the CDC 82 may be indicative of an active value. In certain embodiments, the active values may be any measured capacitance signals 108 below a lower activation threshold 122 or above an upper activation threshold 124. For example, at the time point C 120, the capacitance signals 108 received by the CDC 82 depict decreased capacitance levels below the lower activation threshold 122, resulting from the human tissue 104 (e.g., finger) approaching and/or being within a close proximity of the sensor 12. As a further example, at a time point D 126, the capacitance signals 108 depict decreased capacitance levels below the lower activation threshold 122, and are indicative of physical contact between the capacitance sensor 76 and the human tissue 104. Accordingly, the time point C 120 and the time point D 126 are active values, such as those measured during the time period the medical sensor 12 is “on,” and/or within close proximity/contacting the human tissue 104. With the removal of the human tissue 104 from near the capacitance sensor 76, the capacitance signals 108 may return to ambient levels, as depicted between a time point E 128 and a time point F 130.

As noted above, the activation thresholds 122, 124 may be stored within the CDC 82 and/or the ROM 52 or other suitable computer-readable storage medium. In certain embodiments, the activation thresholds 122, 124 may be associated with certain types of materials, such that a measured capacitance signal 108 may be sensitive to only certain types of materials and/or certain types of human tissue. Accordingly, a measured capacitance signal 108 may not be an active value below and/or above the activation thresholds 122, 124 for a plastic, a metal, and/or a cloth material, but may be below and/or above the activation thresholds 122, 124 for various types of human tissue.

It should be noted that while the illustrated embodiments utilizes various activation thresholds 122, 124 to determine sensor 12 “on”/“off”/“loose” conditions, in other embodiments, the measure capacitance signals 108 (and/or the digital values converted from capacitance signal 108) may be compared with dielectric coefficients of various materials to determine the type material in contact and/or within close proximity of the sensor 12. These dielectric coefficients may be stored in the CDC 82 and/or the ROM 52 or other suitable computer-readable storage medium.

FIG. 6 is a perspective view of an embodiment of the medical sensor 12 of FIG. 2, illustrating a plurality of double-plated capacitance sensors 76 disposed on the sensor body 90, e.g., at different locations on the support layer 94 where the plurality of double-plated capacitance sensors 76 are configured to detect when a portion of the medical sensor 12 loses contact with the human tissue 104. In some situations, a patient monitor 14 may be configured to detect one or more portions of the medical sensor 12 not securely adhered to the human tissue 104 and/or one or more portions of the medical sensor 12 that may lose contact with the human tissue 104 (e.g., fall off) due to the fact that the sensor 12 is improperly applied. For example, the medical sensor 12 may be configured to distinguish between emergency physiological conditions (e.g., asystole) from situations where the sensor 12 loses contact with the human tissue 104. As depicted in the illustrated embodiment, each of the plurality of the double-plated capacitance sensors 76 may be associated with only a portion of the patient-facing surface 92 of the sensor 12. For example, each sensor 76 may be associated with a corner of the sensor body 90. Accordingly, each double-plated capacitance sensor 76 may be configured to detect when that particular portion of the medical sensor 12 loses contact with the human tissue 104. In such embodiments, the signal from each sensor 76 may be processed and the resultant output associated with only a particular region of the sensor body 90, and the output provided by the monitor 14 may indicate an attachment status of individual regions of the sensor body 90.

As described above with respect to FIGS. 2-3, each plate of the double-plated capacitance sensor 76 (e.g., the transmission plate 78 and the receiver plate 80) may be formed on the sensor as a pattern of strips and/or traces from conductive materials, such as copper, carbon, silver, or other similar materials. As illustrated, the double-plated capacitance sensors 76 disposed on the sensor body 90 include the transmission plate 78 and the receiver plate 80 in a rectangular configuration whereby each respective sensor 76 is physically separate (i.e. spaced apart) from the other sensors 76. However, other configurations (e.g. circles, parallel strips, etc.) are contemplated. Further, each double-plated capacitance sensor 76 may be coupled to the CDC 82 via appropriate wires or leads, such that each double-plated capacitance sensor 76 may be configured to receive the excitation signal 106 from the excitation source 4, and provide the capacitance signal 108 to the ADC chip 86 on the CDC 82. In certain embodiments, a timer and/or a GPIO pin having accurate timing information for the capacitor to charge or discharge may be used in lieu of the ADC chip 86. As noted above, based at least in part on the capacitance signal 108 received from the capacitance sensor 76, the patient monitor 14 may be configured to detect the periods of time during which the medical sensor 12 is “on” (e.g., within close proximity/contacting human tissue and/or other materials) and “off” (e.g., not within close proximity/contacting human tissue and/or other materials). For example, the patient monitor 14 may determine when the medical sensor 12 is within close proximity and/or contacting the human tissue 104, and when the medical sensor 12 is not within close proximity and/or contacting the human tissue 104. While the signals may be processed separately to yield separate outputs representative of the contact of individual portions of the sensor body 90, the signals may also be combined or arbitrated between to provide information about the attachment of the sensor body 90 as a whole.

In addition, the patient monitor 14 may be configured to detect when one or more portions of the sensor 12 are not securely adhered and/or fail to maintain contact with the human tissue 104. In certain embodiments, the double-plated capacitance sensors 76 may be disposed along the perimeter of the medical sensor 12, such as at each corner of the sensor 12. For example, as shown in FIG. 6, a first double-plated capacitance sensor 132 may be disposed at an upper right corner 134, and a second double-plated capacitance sensor 136 may be disposed at a lower left corner 138. In certain situations where the upper right corner 134 is not securely applied to the human tissue 104 and/or fails to maintain contact with the human tissue 104 after application (e.g., during sensor 12 operation), the first double-plated capacitance sensor 132 may detect a change in capacitance levels indicative of a sensor “off” situation. Likewise, if the lower left corner 138 is not securely applied to the human tissue 104 and/or fails to maintain contact with the human tissue 104 after application (e.g., during sensor 12 operation), the second double-plated capacitance sensor 136 may detect a change in capacitance levels indicative of a sensor “off” situation.

It should be noted that while the illustrated embodiment depicts the double-plated capacitance sensors 76 disposed along the corners of the medical sensor 12, in other embodiments, the double-plated capacitance sensors 76 may be disposed anywhere on the sensor body 90, including along the perimeter of the sensor 12, as described in detail with respect to FIG. 7. In addition, the plurality of double-plated capacitance sensors 76 may be disposed on the sensor body 90 as an array of capacitance sensors 76, or may be disposed in other configurations (e.g., linear, square, triangular, etc.). Further, while the illustrated embodiment depicts the double-plated capacitance sensors 76, in other embodiments, the single-plated capacitance sensors may be used to detect when one or more portions of the sensor 12 are not securely adhered and/or fail to maintain contact with the human tissue 104, as described further with respect to FIG. 12.

FIG. 7 is a perspective view of the medical sensor 12 of FIG. 2, illustrating a plurality of double-plated capacitance sensors 76 and a water capacitance sensor 140 disposed on the sensor body 90, where the water capacitance sensor 140 is configured to distinguish between water-based fluids and the human tissue 104. In certain embodiments, the capacitance sensors 76 may be include a hydrophobic coating to repel moisture and reduce the effects of moisture on the functionality of the sensors 76. Further, in some situations, the patient monitor 14 may be configured to detect one or more portions of the medical sensor 12 improperly applied to a material other than human tissue 104 (e.g., water, gel, cloth). For example, the medical sensor 12 may be configured to distinguish between water-based fluids (e.g., blood, saline, sweat, alcohol dilutions, hydrogels, etc.) and different types of human tissues 104 (e.g., ear, skin, cartilage, bone, etc.), helping to reduce misreporting of the physiological parameters. As depicted in the illustrated embodiment, each of the plurality of the double-plated capacitance sensors 76 may be associated with a portion of the patient-facing surface 92 of the sensor 12, and may be configured to detect a change in capacitance levels if the portion of the sensor 12 fails to maintain contact with the sensor 12. Further, a water capacitance sensor 140 may be disposed on the medical sensor 12, and may be configured to distinguish between water-based materials and the human tissue 104. In some embodiments, the capacitance sensors 76 may be configured to determine and/or estimate the location of the patient where the sensors 76 are applied based on the capacitance levels measured.

As described above with respect to FIGS. 2-3, 6, and 7, each plate of the double-plated capacitance sensor 76 (e.g., the transmission plate 78 and the receiver plate 80) may be formed on the sensor as a pattern of strips and/or traces from conductive materials, such as copper, carbon, silver, or other similar materials. As illustrated, the double-plated capacitance sensors 76 disposed on the sensor body 90 include the transmission plate 78 and the receiver plate 80 in a double-plated circular configuration. In particular, the double-plated capacitance sensors 76 may be disposed anywhere along the perimeter of the medical sensor 12, such that the double-plated capacitance sensors 76 are configured to detect whether one or more portions of the sensor 12 are not securely applied to the human tissue 104 and/or whether one or more portions of the sensor 12 fail to maintain contact with the human tissue 104 after application (e.g., during sensor 12 operation). In addition, the sensor 12 may include a water capacitance sensor 140 configured to distinguish between water-based materials and the human tissue 104.

In particular, in certain embodiments, the water capacitance sensor 140 may be a double-plated capacitance sensor 76 generally smaller than double-plated capacitance sensors 76 utilized for detection of sensor “on”/“off”/“loose” conditions and/or for detection of sensor losing contact with the tissue. For example, in certain embodiments, the water capacitance sensor 140 may be as small as between approximately 2 mm-5 mm in diameter. In other embodiments, the water capacitance sensor 140 may be 1 mm-2 mm, 5 mm-8 mm, 8 mm-10 mm in diameter, such that the water capacitance sensor 140 may be easily covered in full by a water-based material within close proximity and/or making contact with the sensor 12. Particularly, in situations where the region above the water capacitance sensor 140 is covered in full by a water-based material, the water capacitance sensor 140 may be able to distinguish between water-based materials and the human tissue 104. Indeed, in such situations, a stronger relationship may exist between the capacitance levels and/or change in capacitance levels and the dielectric constants for material types (e.g., water-based materials, cloth, plastics, metals, etc.). For example, when the water capacitance sensor 140 is completely covered by a water-based material, the water capacitance sensor 140 measures a change in capacitance (e.g., capacitance signal 108 provided to the CDC 82) which may utilized to distinguish between the water-based material and/or other types of materials (e.g., types of human tissue, cloth, metal, plastics, etc.).

It should be noted that while the illustrated embodiment describes the use of the smaller double-plated capacitance sensor 76 (e.g., water capacitance sensor 140) as a means for distinguishing between water-based materials and human tissue 104, in other embodiments, the smaller double-plated capacitance sensor 76 may be configured to detect types of materials. For example, a smaller double-plated capacitance sensor 76 has the benefit of being covered in full by a material, such as water-based materials, human tissue types, clothes, metals, plastics, brought within close proximity to the medical sensor 12. Accordingly, in certain embodiments, the capacitance signal 108 provided by the smaller double-plated capacitance sensor 76 may be utilized by the patient monitor 14 to detect the type of material near and/or touching the medical sensor 12. As noted above, the patient monitor 14 may be configured to compare the digital values of the measured capacitance signal 108 with dielectric constants, where each dielectric constant is associated with a different type of material and/or a different type of human tissue. The dielectric constants may vary for different parts of the body and for different types of materials (e.g., water, gel, plastic, cloth, etc.), and may be stored within the CDC 82, within a memory (e.g., a ROM memory 52, a RAM memory 54, non-volatile memory 56, etc.) on the patient monitor 14, and/or within a look-up table.

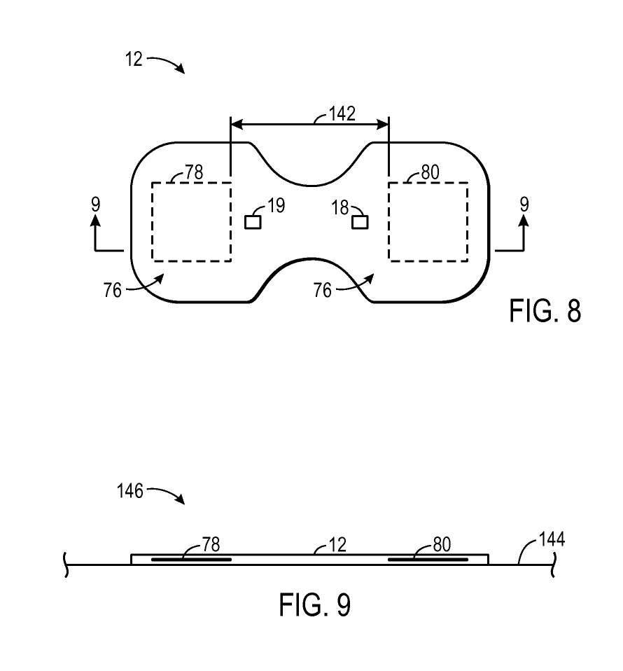

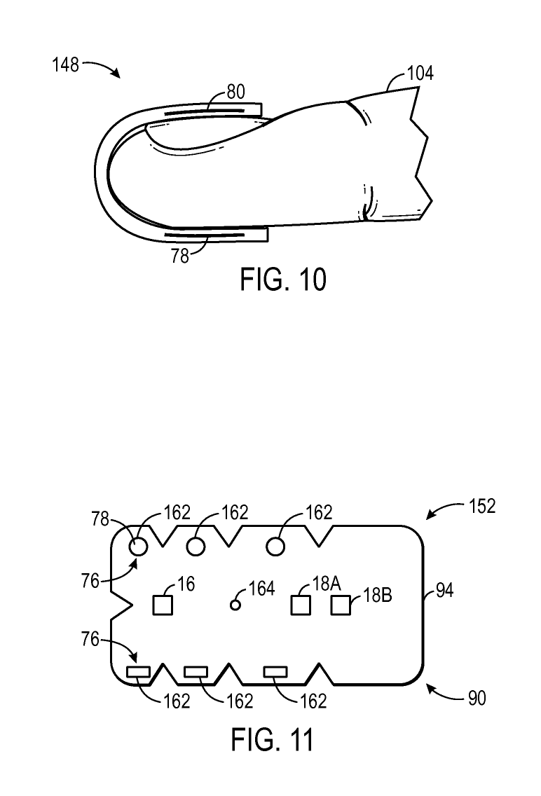

FIG. 8 is a perspective view of the medical sensor 12 of FIG. 2 illustrating the double-plated capacitance sensor 76, where the double-plated capacitance sensor 76 is configured to distinguish between a reflectance mode of sensor 12 operation and a transmission mode of sensor 12 operation. In particular, the illustrated embodiment depicts the double-plated capacitance sensor 76 having the first plate 78 (e.g., transmission plate 78) and the second plate 80 (e.g., receiver plate 80) spaced a first distance 142 apart. The medical sensor 12 may be utilized in various configurations for different modes of patient monitoring. For example, the sensor 12 may be configured in an unfolded orientation 146 for reflectance mode monitoring, such as when the sensor 12 is applied to a patient's forehead 144, as described further with respect to FIG. 9. Further, the sensor 12 may be configured in a folded orientation 148 for transmission mode monitoring, such as when the sensor 12 is applied to a patient's finger 146, as described further with respect to FIG. 10.

FIG. 9 is a perspective view of the medical sensor 12 of FIG. 8, depicting the sensor 12 in the unfolded orientation 146 configured for reflectance mode operation and the medical sensor in the folded orientation 148 configured for transmission mode operation. In certain embodiments, the double-plated capacitance sensor 76 may be configured to detect the configuration and/or orientation of the sensor 12 to determine the mode of sensor 12 operation. Specifically, the double-plated capacitance sensor 76 may utilize the capacitance signal 108 and/or a physiological parameter signal (e.g., a plethysmographic signal, an optical signal, a photoacoustic signal, etc.) to determine the mode of sensor 12 operation. It may be beneficial to determine the mode of sensor 12 operation so that the patient monitor 14 may utilize proper calibration coefficients when processing information received from the sensor 12, such as information related to the change in capacitance levels and/or information related to the physiological parameter signals.

Specifically, in the unfolded orientation 146, the first plate 78 and the second plate 80 of the double-plated capacitance sensor 76 may be configured adjacent to one another, such that the two plates are coplanar. In such configurations, the electric field 102 between the first plate 78 and the second plate 80 may be weak, and the capacitance signal 108 received by the patient monitor 14 may be small or weak. In the folded orientation 148 (shown in FIG. 10), the first plate 78 and the second plate 80 of the double-plated capacitance sensor 76 may be folded over onto opposite sides of the finger 104, such that the two plates are parallel to each other. In such configurations, the electric field 102 between the first plate 78 and the second plate 80 may be more direct and stronger, and the capacitance signal 108 received by the patient monitor 14 may be larger or stronger. In certain embodiments, the patient monitor 14 may be configured to detect the orientation of the sensor 12 based at least in part on the capacitance signal 108 received from the capacitance sensor 76.

In other embodiments, the capacitance signal 108 may be used in conjunction with the physiological parameter signal (e.g., a plethysmographic signal, a photoacoustic signal, photoplethysmograph (PPG) signal, etc.) to determine the mode of operation and/or the sensor 12 configuration. For example, if the patient monitor 14 detects abnormal characteristics within the PPG signal, the patient monitor 14 may be configured to process the capacitance signal 108 to determine the source of the abnormal characteristics. For example, in response to abnormal characteristics detected within the PPG signal, the patient monitor 14 may be configured to detect the type of material (e.g., human tissue, water, gel, cloth, etc.) the medical sensor is applied to via the water capacitance sensor 140, to ensure that all parts of the sensor 12 are properly applied to human tissue 104 instead of other types of materials, to ensure that all parts of the sensor 12 securely maintain contact with the patient's tissue 104, to detect the configuration and/or orientation of the medical sensor 12 on the patient's tissue 104, and/or to determine the mode of operation of the medical sensor 12.

The present techniques (as described with respect to FIGS. 1-10) may be incorporated into various systems that collect and display medical data. By way of example, an INVOS® cerebral/somatic sensor, such as an OxyAlert™ NIR sensor by Covidien, LP., which may include one or more emitters and a pair of detectors for determining site-specific oxygen levels, may represent such sensors. With this in mind, a regional oximetry sensor 152, shown in FIG. 11, may be configured to perform regional oximetry. Indeed, in one embodiment, the regional saturation sensor 152 may be an INVOS® cerebral/somatic sensor available from Covidien, LP. In regional oximetry, by comparing the relative intensities of light received at two or more detectors, it is possible to estimate the blood oxygen saturation of hemoglobin in a region of a body. For example, a regional oximeter may include a sensor to be placed on a patient's forehead and may be used to calculate the oxygen saturation of a patient's blood within the venous, arterial, and capillary systems of a region underlying the patient's forehead (e.g., in the cerebral cortex).

As illustrated in FIG. 11, the regional saturation sensor 152 may include the emitter 16 and the two detectors 18: one detector 18A that is relatively “close” to the emitter 16 and another detector 18B that is relatively “far” from the emitter 16. Light intensity of one or more wavelengths may be received at both the “close” and the “far” detectors 18A and 18B. Thus, the detector 18A may receive a first portion of light and the detector 18B may receive a second portion of light. Each of the detectors 18 may generate signals indicative of their respective portions of light. For example, the resulting signals may be contrasted to arrive at a regional saturation value that pertains to additional tissue through which the light received at the “far” detector 18B passed (tissue in addition to the tissue through which the light received by the “close” detector 18A passed, e.g., the brain tissue) when it was transmitted through a region of a patient (e.g., a patient's cranium). Surface data from the skin and skull is subtracted out to produce a regional oxygen saturation (rSO₂) value for deeper tissues.

In certain embodiments, the regional saturation sensor 152 may also include one or more capacitance sensors 76 configured to measure a change in capacitance resulting from a material, such as, for example, human tissue, water, gel, cloth, and so forth, being placed in close proximity and/or making physical contact with the regional saturation sensor 152. Particularly, the illustrated embodiment depicts a single-plated capacitance sensor 162, having the first plate 78 (e.g., transmission plate 78). As noted above, the first plate 78 of the single-plated capacitance sensor 162 may be configured to function as the transmission plate 78. In such embodiments, the electric field 102 may be formed between the first plate 78 (e.g., transmission plate 78) and a material (e.g., water-based materials, typed of human tissue, plastics, metals, etc.) brought within close proximity of the regional saturation sensor 152, such that the material acts as the second plate (e.g., receiver plate). For example, for the single-plate capacitance sensor 162 having the transmission plate 78, human tissue 104 placed within close proximity and/or having physical contact with the regional saturation sensor 152 may be configured to function as the receiver plate.

The capacitance sensor 76 (e.g., the single-plate capacitance sensor 162) on the regional saturation sensor 152 may be coupled to the CDC 82 on the patient monitor 154. In certain embodiments, a plurality of capacitance sensors 76, such as a plurality of single-plate capacitance sensors 162, are disposed on the regional saturation sensor 152 and may be coupled to the CDC 82 (see FIG. 2). In certain embodiments, the CDC 82 includes the excitation source 84 and the ADC chip 86. The excitation source 84 may be configured to provide the excitation signal 106 to the transmission plate 78, such that the electric field 102 may be formed between the transmission plate 78 and a material within close proximity and/or making physical contact with the single-plate capacitance sensor 162. Further, the single-plate capacitance sensor 162 may provide the capacitance signal 108 to the CDC 82 which converts the capacitance signal 108 into digital values. In certain embodiments the digital values of the converted capacitance signal are provided to the microprocessor 48 for further processing, as described in detail with respect to FIG. 2.

As shown in FIG. 11, a plurality of single-plated capacitance sensors 162 disposed on the sensor body 90, where the plurality of single-plated capacitance sensors 162 are configured to detect when a portion of the sensor 152 loses contact with the human tissue 104. Further, the illustrated embodiment depicts a single-plated water capacitance sensor 164 configured to distinguish between various types of materials (e.g., water-based materials, metals, plastics, etc.) and the human tissue. As noted above with respect to FIGS. 1-10, one or more plates of the capacitance sensor 76 (e.g., the transmission plate 78 and the receiver plate 80) may be formed on the sensor as a pattern of strips and/or traces from conductive materials, such as copper, carbon, silver, or other similar materials. As illustrated, the single-plated capacitance sensors 162 disposed on the sensor body 90 include the transmission plate 78 may be formed out of similar materials, and may be configured to be a circular or rectangular shape. It should be noted that in other embodiments, the single-plated capacitance sensors 162 may be arranged anywhere on the patient-contacting surface of the sensor body 90 in any shape (e.g., square shaped, polygon shapes, triangular configuration, linear configuration, diagonal configuration, etc.) In addition, each of the single-plated capacitance sensors 162 may be coupled to the CDC 82 on the patient monitor 154, such that each single-plated capacitance sensor 162 may be configured to receive the excitation signal 106 from the CDC 82, and provide the capacitance signal 108 to the CDC 82. In particular, the capacitance signal 108 may be indicative of changes in capacitance levels resulting from a material, such as the human tissue 104, coming within close proximity and/or making physical contact with the single-plate capacitance sensor 162 to form the electric field 102.

In certain embodiments, based at least in part on the capacitance signal 108 received from the capacitance sensors 162, the patient monitor 14 may be configured to detect the periods of time during which the sensor 152 is “on” or “loose” (e.g., within close proximity/contacting human tissue and/or other materials or is partially within close proximity/contacting human tissue) and “off” (e.g., not within close proximity or contacting human tissue and/or other materials). For example, the patient monitor 154 may determine when the sensor 152 is within close proximity and/or contacting the human tissue 104, and when the sensor 152 is not within close proximity and/or contacting the human tissue 104. In addition, the patient monitor 154 may be configured to detect when one or more portions of the sensor 152 are not securely adhered and/or fail to maintain contact with the human tissue 104. In certain embodiments, the single-plated capacitance sensors 162 may be disposed along the perimeter of the medical sensor 12. For example, as illustrated, the single-plated capacitance sensors 162 may be disposed along the perimeter of the sensor 152 at corners, or at edges where the risk of the sensor 152 losing contact with the human tissue 104 is greatest. It should be noted that the single-plated capacitance sensors 162 may be arranged in a variety of patterns or configurations (e.g., linear, circular, square, etc.). In some embodiments, when the patient monitor 14 detects that the sensor 152 is “off,” and not within close proximity/contacting human tissue and/or other materials, the monitor 14 may be configured to wait a predetermined period of time (e.g., any number of seconds) before providing an alarm to alert an operator.

In yet other embodiments, the single-plate capacitance sensor 152 may be a single-plate water capacitance sensor 164. The water capacitance sensor 164 may be generally smaller than the single-plate capacitance sensors 162, such that the water capacitance sensor 164 may be easily covered in full by a water-based material within close proximity and/or making contact with the sensor 152. Particularly, in situations where the region above the water capacitance sensor 164 is covered in full by a water-based material, the water capacitance sensor 164 may be able to distinguish between water-based materials and the human tissue 104. Indeed, in certain embodiments, the capacitance signal 108 provided by the smaller single-plated capacitance sensor 162 (e.g., the single-plate water capacitance sensor 164) may be utilized by the patient monitor 154 to detect the type of material near and/or touching the medical sensor 12, such as, for example, water-based materials, human tissue types, clothes, metals, or plastics that cover the smaller single-plate capacitance sensor 164 in full. As noted above with respect to FIG. 7, the patient monitor 154 may be configured to compare the digital values of the measured capacitance signal 108 with dielectric constants, where each dielectric constant is associated with a different type of material and/or a different type of human tissue. The dielectric constants may vary for different parts of the body and for different types of materials (e.g., water, gel, plastic, cloth, etc.), and may be stored within the CDC 82, within a memory (e.g., a ROM memory 52, a RAM memory 54, non-volatile memory 56, as shown in FIG. 2, etc.) on the patient monitor 14, and/or within a look-up table.

The present techniques 12 may also be incorporated into various other systems that collect and display medical data. By way of example, the present techniques may be incorporated into a embodiments of photoacoustic sensors, systems and methods provided for the measurement of various localized physiological parameters, such as parameters related to individual blood vessels or other discrete components of the vascular system. Examples of such parameters may include but are not limited to oxygen saturation, hemoglobin concentration, perfusion, cardiac output, and so forth, for an individual blood vessel.

With this in mind, FIG. 12 is a simplified block diagram of a patient monitoring system 166 configured to be used with a sensor for PA spectroscopy, in accordance with described aspects of the present disclosure. The system 166 includes a photoacoustic spectroscopy sensor 168 (e.g., PA sensor 168) and a monitor 170. During operation, the PA sensor 168 emits spatially modulated light at certain wavelengths into a patient's tissue and detects acoustic shock waves generated in response to the emitted light. The monitor 170 is capable of calculating physiological characteristics based on signals received from the PA sensor 168 that correspond to the detected acoustic shock waves. The monitor 170 may include a display 20 and/or a speaker 21, which may be used to convey information about the calculated physiological characteristics to a user. The PA sensor 168 may be communicatively coupled to the monitor 170 via a cable or, in some embodiments, via a wireless communication link.

In one embodiment, the PA sensor 168 may include the emitter 16 (e.g., a light source 16) and one or more detectors, such as the optical detector 18 and an acoustic detector 172, such as an ultrasound transducer. The present discussion generally describes the use of pulsed light sources to facilitate explanation. However, as noted above, it should be appreciated that the PA sensor 168 may also be adapted for use with continuous wave light sources in other embodiments. Further, in certain embodiments, the light source 16 may be associated with one or more optical fibers for conveying light from one or more light generating components to the tissue site.

The photoacoustic spectroscopy sensor 168 may include the light source 16 and the acoustic detector 172 that may be of any suitable type. For example, in one embodiment, the light source 16 may include one, two, or more light emitting components (such as light emitting diodes) 16 adapted to transmit light at one or more specified wavelengths. In certain embodiments, the emitter 16 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1000 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1000 nm) may be used. In one embodiment, the selected wavelengths of light may penetrate into the tissue of the patient up to approximately 1 cm to approximately 2 cm. In disclosed embodiments that include the emitter 16, it should be understood that the emitter 16 may be coupled to an optical fiber. In other embodiments, the light emitted by the light source 16 may be spatially modulated, such as via a modulator 17.

The emitted light may be intensity modulated at any suitable frequency, such as from 1 MHz to 10 MHz or more. In one embodiment, the emitter 21 may emit pulses of light at a suitable frequency where each pulse lasts 10 nanoseconds or less and has an associated energy of a 1 mJ or less, such as between 1 μJ to 1 mJ. In such an embodiment, the limited duration of the light pulses may prevent heating of the tissue while still emitting light of sufficient energy into the region of interest to generate the desired acoustic waves when absorbed by the constituent of interest.

In one embodiment, the acoustic detector 172 may be one or more ultrasound transducers suitable for detecting ultrasound waves emanating from the tissue in response to the emitted light and for generating a respective optical or electrical signal in response to the ultrasound waves. For example, the acoustic detector 172 may be suitable for measuring the frequency and/or amplitude of the acoustic waves, the shape of the acoustic waves, and/or the time delay associated with the acoustic waves with respect to the light emission that generated the respective ultrasound waves. In one embodiment an acoustic detector 172 may be an ultrasound transducer employing piezoelectric or capacitive elements to generate an electrical signal in response to acoustic energy emanating from the tissue of the patient, i.e., the transducer converts the acoustic energy into an electrical signal.

In some embodiments, the system 166 may also include any number or combination of additional medical sensors (e.g., the sensor 12, the regional saturation sensor 152, etc.) or sensing components for providing information related to patient parameters that may be used in conjunction with the PA spectroscopy sensor 168. For example, suitable sensors may include sensors for determining blood pressure, blood constituents, respiration rate, respiration effort, heart rate, patient temperature, cardiac output, and so forth. Such information may be used, for example, to determine if the patient is in shock or has an infection.

In one embodiment, the photoacoustic sensor 168 may include a memory or other data encoding component, depicted in FIG. 2 as an encoder 47. For example, the encoder 47 may be a solid state memory, a resistor, or combination of resistors and/or memory components that may be read or decoded by the monitor 170, such as via reader/decoder 49, to provide the monitor 170 with information about the attached sensor 168. For example, the encoder 47 may encode information about the sensor 168 or its components (such as information about the light source 16 and/or the acoustic detector 172). Such encoded information may include information about the configuration or location of photoacoustic sensor 168, information about the type of lights source(s) 16 present on the sensor 168, information about the wavelengths, pulse frequencies, pulse durations, or pulse energies which the light source(s) 16 are capable of emitting, information about the nature of the acoustic detector 172, and so forth. This information may allow the monitor 170 to select appropriate algorithms and/or calibration coefficients for calculating the patient's physiological characteristics, such as the amount or concentration of a constituent of interest in a localized region, such as a blood vessel.

In one embodiment, signals from the acoustic detector 172 (and decoded data from the encoder 47, if present) may be transmitted to the monitor 170. The monitor 170 may include data processing circuitry (such as one or more processors 48, application specific integrated circuits (ASICS), or so forth) coupled to an internal bus 50. Also connected to the bus 50 may be a ROM memory 52, the RAM memory 54, the speaker 21 and/or a display 20. In one embodiment, a time processing unit (TPU) 58 may provide timing control signals to light drive circuitry 60, which controls operation of the light source 16, such as to control when, for how long, and/or how frequently the light source 16 is activated, and if multiple light sources are used, the multiplexed timing for the different light sources.

TPU 58 may also control or contribute to operation of the acoustic detector 172 such that timing information for data acquired using the acoustic detector 172 may be obtained. Such timing information may be used in interpreting the shock wave data and/or in generating physiological information of interest from such acoustic data. For example, the timing of the acoustic data acquired using the acoustic detector 172 may be associated with the light emission profile of the light source 16 during data acquisition. Likewise, in one embodiment, data acquisition by the acoustic detector 172 may be gated, such as via a switching circuit 64, to account for differing aspects of light emission. For example, operation of the switching circuit 64 may allow for separate or discrete acquisition of data that corresponds to different respective wavelengths of light emitted at different times.

In one embodiment, the received signal from the acoustic detector 172 may be amplified (such as via amplifier 66), may be filtered (such as via filter 68), and/or may be digitized if initially analog (such as via an analog-to-digital converter 70). The digital data may be provided directly to the processor 48, may be stored in the RAM 54, and/or may be stored in a queued serial module (QSM) 72 prior to being downloaded to RAM 54 as QSM 72 fills up. In one embodiment, there may be separate, parallel paths for separate amplifiers, filters, and/or A/D converters provided for different respective light wavelengths or spectra used to generate the acoustic data.

The data processing circuitry (such as processor 48) may derive one or more physiological characteristics based on data generated by the photoacoustic sensor 170. For example, based at least in part upon data received from the acoustic detector 172, the processor 48 may calculate the amount or concentration of a constituent of interest in a localized region of tissue or blood using various algorithms. In one embodiment, these algorithms may use coefficients, which may be empirically determined, that relate the detected acoustic waves generated in response to pulses of light at a particular wavelength or wavelengths to a given concentration or quantity of a constituent of interest within a localized region.

In one embodiment, processor 48 may access and execute coded instructions from one or more storage components of the monitor 170, such as the RAM 54, or the ROM 52. For example, code encoding executable algorithms may be stored in a ROM 52 or the memory 56 (such as a magnetic or solid state hard drive or memory or an optical disk or memory) and accessed and operated according to processor 48 instructions. Such algorithms, when executed and provided with data from the sensor 168, may calculate a physiological characteristic as discussed herein (such as the concentration or amount of a constituent of interest). Once calculated, the physiological characteristic may be displayed on the display 20 for a caregiver to monitor or review.

In certain embodiments, the PA sensor 168 may include one or more capacitance sensors 76, such as the illustrated single-plate capacitance sensors 162, 164. In other embodiments, the PA sensor 168 may include double-plate capacitances sensors 76 or a combination of single-plate sensors 162 and double-plate sensors 76. The capacitance sensors 76 may be coupled to the CDC on the patient monitor 170, such that each capacitance sensor 76 may be configured to receive the excitation signal 106 from the CDC 82, and provide the capacitance signal 108 to the CDC 82. Specifically, the capacitance sensors 76 may be disposed on the PA sensor 168 separate from the acoustic transmitting areas (e.g., areas surrounding the light source 16 and/or the modulator 17) and detecting areas (e.g., areas surrounding the detectors 18, 172), so that the electric field 102 proximal to the capacitance sensors 76 do not interfere with the PA methods and techniques.

In certain embodiments, based at least in part on the capacitance signal 108 received from the capacitance sensors 76, the patient monitor 170 may be configured to detect the periods of time during which the sensor 168 is “on” (e.g., within close proximity/contacting human tissue and/or other materials) and “off” (e.g., not within close proximity/contacting human tissue and/or other materials). In addition, the patient monitor 170 may be configured to detect when one or more portions of the sensor 168 are not securely adhered and/or fail to maintain contact with the human tissue 104. Further still, the patient monitor 170 may be configured to detect the type of material near and/or touching the medical sensor 168, such as, for example, water-based materials, human tissue types, clothes, metals, or plastics. Specifically, the patient monitor 170 may be configured to determine the type of human tissue the sensor 168 is applied to, such forehead skin, temple skin, finger, etc. In addition, based on the capacitance signal 108 and/or the signals received from the acoustic detector 172 or the optical detector 18, the patient monitor 170 may be configured to detect the orientation and/or the configuration of the sensor 168 as it is applied to the human tissue.

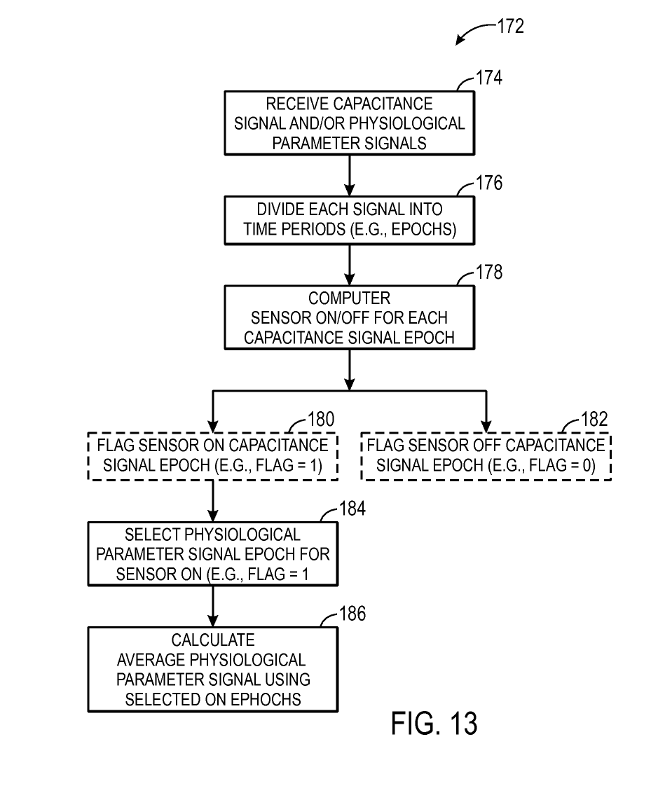

FIG. 13 is a flow chart of a method 172 for calculating a physiological parameter signal based at least in part on the capacitance signal 108 received by the capacitance sensors 76 of FIGS. 1-12. As noted above, the capacitance sensors 76 provide the capacitance signal 108 to the CDC 82 on the patient monitor 14 (e.g., the monitor 14, 154, 170), based on changes in capacitance levels resulting from a material within close proximity and/or touching the medical sensors 12 (e.g., the sensor 12, 152, 168). In certain embodiments, the capacitance signal 108 may be used to determine periods of time during which the sensor 12 is active and “on,” such as during periods of time when the sensor 12 is physically contacting the patient's tissue. Indeed, one or more capacitance sensors 76 may be used to determine sensor “on” or “loose” periods, such as in embodiments where each capacitance sensor 76 is utilized to determine if a portion of the sensor 12 is peeling and/or fails to maintain contact with the patient's tissue (e.g., poorly applied sensor 12). Further, the capacitance signal 108 may be used to determine periods of time during which the sensor 12 is “off,” such as during periods of time when the sensor 12 is non-active, “off,” and/or not within close proximity of the patient's tissue. Accordingly, in certain embodiments, the patient monitor 14 may be configured to determine and average the physiological parameter signal during the one or more periods of time where the sensor 12 is “on” and fully maintaining contact with the patient's tissue.

With the forgoing in mind, FIG. 13 illustrates the method 172 utilized to calculate the average the physiological parameter signal during the one or more periods of time where the sensor 12 is “on,” active, and receives physiological parameter information. The method 172 begins with the patient monitor 14 receiving the capacitance signal 108 and the one or more physiological parameter signals (block 174). The physiological parameter signal may be any signal collected by the patient monitoring system, such as for example, the plethysmographic signal, the optical signal, the photoacoustic signal, the photoplethysmograph (PPG) signal, and so forth. As noted above, the physiological parameter signals may be used to determine one or more physiological parameters related to individual blood vessels or other discrete components of the vascular system. Examples of such parameters may include but are not limited to oxygen saturation, hemoglobin concentration, perfusion, cardiac output, blood pressure, blood constituents, respiration rate, respiration effort, heart rate, patient temperature, and so forth, for an individual blood vessel. In certain situations during the operation of the sensor 12, the patient monitor 14 may not receive a physiological parameter signal or may not receive a physiological parameter signal that accurately represents the physiological conditions of the patient's tissue. For example, as noted above, the capacitance signal 108 may be used to detect abnormal sensor 12 activities, such as detect periods of time during which the sensor 12 is not attached to human tissue or is in an incorrect mode of operation. Accordingly, the patient monitor 14 may also be configured to determine periods of time during which the patient monitor 14 is attached to human tissue and receives accurate physiological parameter signals, and may be configured to average those periods of time, as described further below. It should be noted that in certain embodiments, one or more capacitance signals 108 may be averaged used the steps of methods 172.

The method 172 further includes dividing the capacitance signal 108 and the one or more physiological parameter signals received by the patient monitor 14 into equal sized time periods, or epochs (block 176). For example, each signal may be divided into 100 equal epochs, 150 equal epochs, 200 equal epochs, or any other suitable number of epochs. Depending on the signal, the epoch may be representative of a time period of 1 to 5 seconds, 5 to 9 seconds, 10 to 15 seconds, 16 to 20 seconds, 20 to 40 seconds, and so forth. The number and duration of the epochs may be pre-determined and stored within the memory of the patient monitor 14, or may be provided through user inputs 22. It should be noted that in other embodiments, the method 172 includes dividing the capacitance signal 108 and the one or more physiological parameter signals received by the patient monitor 14 into epochs of different sized time periods, such that each epoch is representative of a different time period between 1 to 5 seconds, 5 to 9 seconds, 10 to 15 seconds, 16 to 20 seconds, 20 to 40 seconds, and so forth. Indeed, any method of division may be utilized to divide the capacitance signal 108 and the one or more physiological parameter signals, so long as the signals are split into a manageable and/or a desired period of time. In some embodiments, user input may be utilized to select one or more pieces of the signals as the epochs of the signal.

In certain embodiments, the patient monitor 14 may determine whether the sensor 12 is “on”/“off”/“loose” for each epoch of the capacitance signal 108 (block 178). In such embodiments, the patient monitor 14 may be configured to utilize the capacitance value (e.g., measured and converted capacitance signal 108) for each epoch of the capacitance signal 108 to determine whether the sensor 12 was “on” or “off” For example, the CDC 82 may be configured to receive the capacitance signal 108 and convert the capacitance signal 108 into digital values (e.g., capacitance value). The patient monitor 14 may process and analyze the capacitance value using one or more algorithms. Specifically, the patient monitor 14 may be configured to analyze the capacitance value for each epoch of the capacitance signal 108. The microprocessor 48 may be configured to compare the capacitance value with one or more coefficients, constants, thresholds, limits, and/or values, which may be empirically determined or which may be pre-loaded and stored in the ROM 52 or other suitable computer-readable storage medium (e.g., RAM 54, memory 56). The capacitance value may be compared to an upper activation threshold and/or a lower activation threshold representative of the threshold values beyond which the sensor is “on.” For example, when the capacitance value exceeds either the upper activation threshold or the lower activation threshold, the measured change in capacitance may be considered to be an active value representing a sensor “on” condition. In other words, the measured capacitance value may be below a lower activation threshold, and may be associated with the capacitance sensor 76 responding to a decrease in capacitance resulting from human tissue being in close proximity and/or contact with the sensor 12. Accordingly, the patient monitor 14 may be configured to determine whether the sensor is “on” or “off” during each epoch of the capacitance signal 108 based at least in part on the capacitance values of each epoch.

In certain embodiments, the patient monitor 14 may determine that the sensor 12 was “on” during a particular period of time based on the capacitance signal 108. For example, the patient monitor 14 may calculate a binary flag corresponding to each epoch of the capacitance signal 108 associated with the sensor “on” condition. In such situations, the patient monitor 14 may be configured to flag the detected sensor “on” epoch with a binary flag (e.g., flag=1) (block 180). Likewise, the patient monitor 14 may calculate a binary flag corresponding to each epoch of the capacitance signal 108 associated with the sensor “off” condition. In such situations, the patient monitor 14 may be configured to flag the detected sensor “off” epoch with a binary flag (e.g., flag=0) (block 182). In this manner, each epoch of the capacitance signal 108 may be flagged as either sensor “on” or sensor “off” and calculations of sensor “on”/“off” may be repeated for each capacitance signal 108 epoch.

The method 172 further includes selecting the portions of the physiological parameter signal that correspond to the capacitance signal 108 epochs having a sensor “on” binary flag (e.g., flag=1) (block 184). In certain embodiments, the selected portions of the physiological parameter signal are averaged to generate an averaged physiological parameter signal. The averaged physiological parameter signal may be used to determine physiological parameters of interest.

For example, the method 172 may be applied to the pulse oximetry techniques described above, and also may be applied to the photoacoustic techniques described in FIG. 12. The capacitance sensors 76 may be configured to provide one or more capacitance signals 108 to the CDC 82, while the optical detector 18 and/or the acoustic detector 172 may be configured to provide one or more physiological parameter signals to the monitor 170. The patient monitor 170 may be configured to divide each signal received into an equal number of epochs of the same duration. For example, each of the capacitance signals 108 and each of the physiological parameter signals may be divided into 100 epochs, with each epoch having a 10 nanosecond duration. For each capacitance signal 108 epoch, the patient monitor 170 may be configured to determine whether the sensor 168 was “on,” and may be configured to binary flag (flag=1) the capacitance signal 108 for sensor “on” epochs. In certain embodiments, the patient monitor 170 may be configured to select portions of the physiological parameter signals corresponding to the sensor “on” epoch of the capacitance signal 108. Further, the patient monitor 170 may be configured to calculate an average physiological parameter signal of PA signal using the selected portions. From each averaged PA signal, the patient monitor 170 may be configured to calculate (e.g., peak-to-peak measurements) parameters of interest, which may translate to one or more points on an ID curve. This may be repeated for each averaged PA signal to determine a cardiac output estimation.

While the disclosure may be susceptible to various modifications and alternative forms, specific embodiments have been shown by way of example in the drawings and have been described in detail herein. Further, specific elements of the disclosed embodiments may be combined or exchanged with one another. It should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A sensor, comprising:
an emitter disposed on a tissue-facing surface of the sensor and configured to emit one or more wavelengths of light into a tissue region of a patient;
a detector disposed on the tissue-facing surface of the sensor;
a capacitive element comprising at least one plate configured to generate and measure an electric field; and
a non-conductive material disposed on the tissue-facing surface of the sensor, wherein the non-conductive material is configured to separate the capacitive element from the tissue region of the patient.

2. The sensor of claim 1, wherein the detector is an acoustic ultrasound detector configured to detect acoustic energy generated by the tissue region of the patient in response to the one or more emitted wavelengths of light.

3. The sensor of claim 1, wherein the detector is a photodetector configured to receive the one or more emitted wavelengths of light.

4. The sensor of claim 1, wherein the capacitive element is a single plate comprising a first plate configured to generate a voltage potential (AC/DC) and measure a current flow or charge times into the patient impedance.

5. The sensor of claim 1, wherein the capacitive element is a two plate configuration comprising a first plate and a second plate configured to generate a signal communicatively coupled to a capacitance-to-digital converter.

6. The sensor of claim 1, wherein the capacitive element is a three plate configuration comprising a first plate, a second plate, and a third plate, wherein a first capacitance signal corresponding to a first tissue site is generated based on the first plate and the second plate and a second capacitance signal corresponding to a second tissue site is generated based on the first plate and the third plate, and wherein a portion of the first tissue site overlaps with the second tissue site.

7. The sensor of claim 5, wherein the first plate of the capacitive element is a transmission plate configured to transmit an excitation signal from an excitation signal source.

8. The sensor of claim 5, wherein the second plate of the capacitive element is a receiver plate configured to receive the signal from the capacitance-to-digital converter on the patient monitor.

9. The sensor of claim 5, wherein the first plate and the second plate are separated by a first distance, and wherein the emitter and the detector are disposed in between the first plate and the second plate on the tissue-contacting surface of the sensor.

10. The sensor of claim 5, wherein the first plate and the second plate are formed as one or more traces of conductive material.

11. The sensor of claim 1, comprising one or more capacitive elements disposed along a perimeter of the tissue-facing surface of the sensor.

12. The sensor of claim 9, wherein each of the one or more capacitive elements are configured to generate an output signal based on the strength of the electric field, and wherein the output of the each of the one or more capacitive elements is utilized to determine if the sensor fails to maintain contact with the tissue region of the patient.

13. A monitoring system, comprising:
an input for a plethysmographic signal responsive to light emitted into a tissue of a patient and a capacitance signal based on a strength of an electric field applied by a single plate to the tissue;
a monitor coupled to the input, wherein the monitor comprises:
a memory storing instructions for:
receiving the plethysmographic signal;
receiving the capacitance signal; and
determining if a sensor is associated with the tissue based at least in part on the capacitance signal, wherein the capacitance signal is representative of a capacitance level of the electrical field generated between the first plate and the tissue; and
a processor configured to execute the instructions.

14. The system of claim 13, wherein determining if the sensor is associated with the tissue comprises determining if the capacitance signal is higher than an upper activation threshold.

15. The system of claim 14, wherein the upper activation threshold comprises a dynamic threshold.

16. The system of claim 13, wherein determining if the sensor is associated with the tissue comprises determining a measure of a variance of the capacitance signal, and comparing the variance measure to a threshold.

17. The system of claim 13, wherein determining if the sensor is associated with the tissue comprises determining if the capacitance signal is lower than a lower activation threshold.

18. The system of claim 17, wherein the lower activation threshold comprises a dynamic threshold.

19. The system of claim 13, comprising two or more single plates configured to generate respective electric fields when applied to the tissue, and wherein each single plate is configured to provide respective capacitance signals to a capacitance-to-digital converter on the monitor.

20. The system of claim 19, wherein each of the single plates are configured to generate respective capacitance signals based on the strength of the electric field, and wherein the capacitance signals of the each of the two or more single plates are utilized to determine if the sensor is associated with the tissue.

21. The system of claim 13, wherein determining if the sensor is associated with the tissue comprises distinguishing between the tissue and a water-based material based on the capacitance signal.

22. The system of claim 13, wherein determining if the sensor is associated with the tissue comprises distinguishing between transmission mode and reflectance mode of sensor operation.

23. A method, comprising:
receiving a capacitance signal from a capacitive element disposed on a patient sensor, wherein the capacitive element is configured to generate a capacitance signal based on the strength of an electric field;
receiving a physiological parameter signal from the sensor;
dividing each signal into epochs comprising corresponding to time periods of the physiological parameter signal and the capacitance signal;
determining whether each epoch of the capacitance signal are associated with a sensor on or sensor off condition; and
determining the physiological parameter based on the epochs of the physiological parameter signal associated with a sensor on capacitance signal.

24. The method of claim 23, wherein determining a sensor on condition comprises determining if the capacitance signal is higher than an upper activation threshold.

25. The system of claim 23, wherein determining the physiological parameter comprises determining an average photoacoustic signal from an acoustic detector.
